(19) United States
(12) Patent Application Publication
(10) Pub. No.: US 2008/0268068 A1
(43) Pub. Date: Oct. 30, 2008

(54) Sparc Promoter Mutations Associated with Drug Resistance, and Methods, Oligonucleotides, Cells and Arrays Associated Therewith

(76) Inventor: Isabella T. Tai, Vancouver (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/791,875

(22) PCT Filed: Nov. 30, 2005
(86) PCT No.: PCT/CA2005/001819

§ 371 Date: Apr. 21, 2008

Related U.S. Application Data
(60) Provisional application No. 60/631,414, filed on Nov. 30, 2004.

(51) Int. Cl. C12Q 1/68; C40B 30/04; C40B 40/06; C07H 21/00; C12N 15/85; C12N 5/06; C07K 2/00; A61K 33/24; A61P 35/00

(52) U.S. Cl. 424/649; 435/6; 506/9; 506/16; 536/24.31; 536/23.1; 435/320.1; 435/325; 530/300; 506/18

(57) ABSTRACT

The invention provides oligonucleotide or peptide nucleic acids, peptide nucleic acids, arrays, cells, methods and kits for determining the presence or absence of a SPARC promoter mutation indicative of a cancer's resistance or sensitivity to a therapeutic regimen. The method generally comprises determining presence or absence of a SPARC promoter mutation for a subject's cancer. The invention also provides for methods of identifying potential subjects having a resistant cancer for selection to administer an alternative therapy regimen. The invention also provides for methods of treating such subjects with a therapeutic regimen based on the subject's genotype.

### FIELD OF THE INVENTION

The field of the invention relates to the assessment and/or treatment of an animal with a neoplastic condition.

### BACKGROUND OF THE INVENTION

Resistance to chemotherapy is common to many types of cancer and contributes to the high mortality rates in cancer patients. Many factors can play a part in the initial intrinsic resistance to therapy, such as upregulation of efflux pumps from multidrug resistance (MDR) family P-glycoprotein and other MDR proteins (for example multi-drug resistance-associated protein MRP). Such efflux pumps remove chemotherapeutic agents and their metabolites out of cells, thereby decreasing the efficacy of the chemotherapeutic regimen. However, not all cancers that are resistant to chemotherapy have high expression levels of MDR proteins, suggesting that there may be other mechanisms for therapeutic resistance.

Some cancers are particularly resistant to therapies. For example, colorectal cancer (CRC), often accumulates numerous genetic mutations in the progression to tumorigenesis that can also contribute to drug resistance (mutations in p53; loss of DNA mismatch repair (MMR) genes associated with an increased rate of mutation to drug resistance in hereditary non-polyposis colorectal cancer (HNPCC); K-ras mutations; loss of heterozygosity of chromosomes 18q and 22q; and mutations in cell cycle regulatory genes, p21 and p27, is associated with preventing apoptosis in tumors following some chemotherapeutic treatments).

The secreted protein, acidic, rich in cysteine (SPARC), also known as osteonectin, BM-40 and 43K protein, is a type of extracellular protein, termed a matricellular protein, having counter-adhesive properties shown to disrupt cell-matrix interactions (BORNSTEIN P. J. Cell Biol (1995) 130:503-506; SAGE E H. and BORNSTEIN P. J Biol Chem. (1991) 266:14831-4). SPARC is a Ca²⁺-binding glycoprotein, which maps to chromosome 5q31-q33 (SWAROOP A. et al. Genomics (1988) 2:37-47), the genomic sequence is reported to have 10 exons and 9 introns (VILLARREAL X C. et al. Biochemistry (1989) 28(15):6483-6491) with the cDNA extending over ˜3 kb. Representative Homo sapiens SPARC sequences are listed in GenBank under accession numbers NM_{—}003118.2 (gi48675809-mRNA), J03040 (SWAROOP A. et al. (1988) supra) and J02863 (VILLARREAL X C. et al. (1989) supra). A representative sequence containing the SPARC promoter, submitted by JENDRASCHAK E. and KAMINSKI W E. (Genomics (1998) 50(1):53-60), is U65081.1 (gi3253140) containing 1370 bp. The SPARC promoter region (accession number X82259) was characterized using various promoter constructs in a luciferase assay by HAFNER M. et al. (Matrix Biology (1994) 14:733-741).

SPARC has been associated with bone mineralization (TERMINE J D. et al. Cell (1981) 26:99-105), tissue remodeling (TREMBLE P M. et al. J. Cell Biol. (1993) 121:1433-1444), endothelial cell migration (SAGE E H. et al. J. Cell Biol. (1989) 109:341-356; HASSELAAR P. and SAGE E H. J. Cell Biochem. (1992) 49(3):272-283), apoptosis (SHI Q. et al. J. Biol. Chem. (2004) 279(50):52200-09), angiogenesis (KUPPRION C. et al. J. Cell Biol. (1998) 273(45):29635-29640; VAJKOCZY P. et al. Int. J. Cancer (2000) 87:261-268), scleroderma (LAGAN A L. et al. Rheumatology (2005) 44(2):197-201; ZHOU X. et al. Arthritis & Rheumatism (2002) 46(11):2990-2999) and adipose tissue accumulation (BRADSHAW A D. et al. PNAS (2003) 100(10):6045-6050). Furthermore, SPARC has also been associated with poor overall survival in head and neck cancer (HNSCC) patients (CHIN D. et al. Int. J. Cancer (2005) 113:789-797), poor prognosis in human breast cancer (JONES C. et al. Cancer research (2004) 64:3037-3045), tumor suppressor activity in human ovarian carcinoma cells (MOK S C. et al. Oncogene (1996) 12:1895-1901), development of squamous cell carcinomas in mice (AYCOCK R L. et al. J. Invest. Dermatol. (2004) 123:592-599), inhibition of Lewis lung carcinoma (LLC) cells (BREKKEN R A. et al. J. Clin. Invest. (2003) 111:487-495) and advanced stage human gastric carcinomas (WANG C-S. et al. British Journal of Cancer (2004) 91:1924-1930).

International application, WO 2004/064785, describes methods of sensitizing mammalian cancers to a therapeutic treatment through administration of the SPARC family polypeptides or polynucleotides encoding a SPARC polypeptide.

### SUMMARY OF THE INVENTION

This invention is based in part on the surprising discovery that SPARC promoter mutations are associated with therapeutic resistance in a variety of cells and to a variety of therapeutic agents.

This invention is also based in part on the identification the particular mutations in (SPARC) sequence associated with resistance to therapeutic regimens.

In accordance with one aspect of the invention, methods are provided for predicting responsiveness of a subject to a therapeutic regimen, the subject having, or suspected of having a cancer, the method including determining a presence or an absence of one or more a secreted protein, acidic, cysteine-rich (SPARC) promoter mutations in the subject's cancer, wherein said presence or absence of one or more SPARC promoter mutations is indicative of a sensitivity of the subject's cancer to the therapeutic regimen.

In accordance with another aspect of the invention, there is provided a kit for detecting a mutation within a SPARC promoter in a subject's cancer to provide an indication of the subject's sensitivity to a therapeutic regimen, the kit may include: a restriction enzyme capable of distinguishing alternate nucleotides at the mutation site; or a labeled oligonucleotide having sufficient complementary to the mutation site so as to be capable of selectively hybridizing to either the mutated or non-mutated site. The kit may further include an oligonucleotide or a set of oligonucleotides operable to amplify a region including the mutation. The kit may also include a polymerization agent and/or instructions for using the kit to identify mutations in a SPARC promoter sequence.

In accordance with another aspect of the invention, methods are provided for selecting a group of subjects for determining the efficacy of a therapeutic regimen known or suspected of being useful for the treatment of cancer, the method including determining a genotype at one or more mutation sites in a SPARC promoter for each subject, wherein said genotype is indicative of the subject's sensitivity to a therapeutic regimen. The method may further include, administering the therapeutic regimen to the subjects or a subset of subjects and determining each subject's sensitivity to the therapeutic regimen. The method may further include comparing subject response to the therapeutic regimen based on genotype of the subject.

In accordance with another aspect of the invention, oligonucleotides or peptide nucleic acids of about 10 to about 400 nucleotides are provided that may be used in the identification of mutations and that may hybridize specifically to a sequence contained in a human target sequence consisting of a subject's SPARC promoter sequence, a complementary sequence of the target sequence or RNA equivalent of the target sequence to and wherein the oligonucleotide or peptide nucleic acid is operable in determining the presence or absence of a SPARC promoter mutation. The oligonucleotides or peptide nucleic acids may alternatively be of about 15 to about 300 nucleotides, about 20 to about 200 nucleotides.

In accordance with another aspect of the invention, a nucleic acid is provided including a nucleic acid corresponding to SEQ. ID NO: 1 with one or more of the following mutations relative to SEQ ID NO: 1: (a) 295delG and 301A>CC; (b) 315insA and 316G>A; (c) 341C>T, 342T>C, 344A>C, 347C>A, 354G>A, 356G>A, 359T>G and 363G>A; (d) 395C>T and 396A>T; (e) 488T>C; (f) 533A>G; (g) 734G>A; (h) 734delG; (i) 769T>A, 771G>C and 774G>C; (j) 784T>C, 786T>G and 788T>A; (k) 793T>G, 796T>A and 800T>C; (l) 820C>A and 824T>A; (m) 864T>A and 867T>A; (n) 895T>G, 899T>A, 901G>A and 904G>A; (o) 901G>A; (p) 901G>A, 904G>A, 929A>G and 934delC; (q) 922T>G, 928T>G and 935T>G; (r) 982T>C, 984T>G, 985G>A, 991G>A, 995G>A, 999C>T and 1000T>G; (s) 1063G>C; (t) 1079G>A; (u) 1085delC, 1086delA, 1087delG, 1089G>A, 1090T>C, 1091T>C and 1092G>T; (v) 1161C>A, 1162T>C, 1163delA and 1164delT; and (w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T. The mutation may result in altered levels of intracellular and/or extracellular levels of a polypeptide encoded by the SPARC sequence. Altered levels as used herein may be a reduction in the intracellular and/or extracellular SPARC polypeptide. The nucleic acid may further include a vector. In accordance with another aspect of the invention a cell is provided including the nucleic acids described herein.

In accordance with another aspect of the invention, oligonucleotides or peptide nucleic acids are provided that may be used in the identification of SPARC sequence mutations in accordance with the methods described herein, the oligonucleotides or peptide nucleic acids are characterized in that the oligonucleotides or peptide nucleic acids hybridize under normal hybridization conditions with a region of one of sequences identified by SEQ ID NO: 1-33, etc. or their complements to determine the presence or absence of one or more SPARC promoter mutations within a target sequence.

In accordance with another aspect of the invention, an oligonucleotide primer is provided including a portion of SEQ ID NO: 1-33 or their complements, wherein said primer is 12 to 54 nucleotides in length and wherein the primer specifically hybridizes to a region of SEQ ID NO: 1-33 or their complements and is capable of identifying SPARC sequence mutations described herein. Alternatively, the primers may be between sixteen to twenty-four nucleotides in length.

In accordance with another aspect of the invention, oligonucleotide or peptide nucleic acids are provided of about 10 to about 400 nucleotides that hybridize specifically to a sequence contained in a human target sequence including SEQ ID NO: 1-33, a complementary sequence of the target sequence or RNA equivalent of the target sequence and wherein the oligonucleotide or peptide nucleic acid is operable in determining a mutation at one or more of positions of the SPARC promoter sequence as described herein.

In accordance with another aspect of the invention arrays of nucleic acid molecules attached to a solid support are provided, the array may include one or more oligonucleotides or peptide nucleic acids as described herein.

In accordance with another aspect of the invention an array of nucleic acid molecules attached to a solid support is provided, the array may include an oligonucleotide or peptide nucleic acids that may specifically hybridize to a nucleic acid molecule having one or more of the mutations set out in TABLE 1.

The oligonucleotide or peptide nucleic acid may further include one or more of the following: a detectable label; a quencher; a mobility modifier; a contiguous non-target sequence situated 5′ or 3′ to the target sequence or 5′ and 3′ to the target sequence.

In accordance with another aspect of the invention a computer readable medium is provided, wherein the medium may have a plurality of digitally encoded mutation correlations selected from the SPARC mutation correlations in TABLE 4, wherein each correlation of the plurality has a value representing an indication of responsiveness to treatment of a cancer with a therapeutic regimen.

In accordance with another aspect of the invention methods are provided for subject screening whereby the method includes the steps of (a) selecting a subject based mutations in the SPARC sequence indicative of resistance to a therapeutic regimen, (b) obtaining SPARC sequence information from the subject and (c) detecting the one or more mutations in the SPARC sequence, wherein the mutation is indicative of the resistance a subject's cancer to the therapeutic regimen.

In accordance with another aspect of the invention, methods are provided for selecting a group of subjects to determine the efficacy of a candidate drug known or suspected of being useful for the treatment of a cancer, the method including detecting one or more mutations in a SPARC sequence for each subject, wherein said one or more mutations are indicative of the subject's response to a therapeutic regimen. The method may also include administering the candidate cancer drug to the subjects or a subset of subjects and determining each subject's response to the therapeutic regimen. The method may also include the additional step of comparing subject response to the candidate drug based on the one or more mutations in the subject's SPARC sequence. Response to the candidate drug may be decided by determining each subject's ability to recover from the cancer or by determining the progression of the cancer in response to the therapeutic regimen.

In accordance with another aspect of the invention, methods are provided for selecting a subject for the treatment of a cancer with a therapeutic regimen, including the step of identifying a subject having one or more mutations in their SPARC sequence, wherein the identification of a subject with the one or more mutations is predictive of decreased responsiveness to the therapeutic regimen.

In accordance with another aspect of the invention, methods are provided for treating a cancer in a subject, the method including administering a therapeutic regimen to the subject, wherein said subject does not have one or more of the SPARC sequence mutations indicative of resistance to the therapeutic regimen. Alternatively, subjects having one or more of the SPARC sequence mutations indicative of resistance to the therapeutic regimen may be treated with an alternative treatment regimen. The alternative treatment regimen may include the administration of SPARC polypeptides.

In accordance with another aspect of the invention, the use of a therapeutic regimen in the manufacture of a medicament for the treatment of a cancer, wherein the subjects treated do not have a mutation indicative of resistance to the therapeutic regimen in their SPARC promoter sequence.

In accordance with another aspect of the invention, the use of a therapeutic regimen in the manufacture of a medicament for the treatment of the cancer in a subset of subjects, wherein the subset of subjects do not have a mutation indicative of resistance to the therapeutic regimen in their SPARC promoter sequence.

In accordance with another aspect of the invention, there is provided a use of an alternative therapeutic regimen for treating a cancer in a subject, for the manufacture of a medicament, the use comprising, selecting a subject having a mutation in a SPARC promoter sequence; and administering to said subject the alternative therapeutic regimen.

In accordance with another aspect of the invention, methods are provided for detecting a mutation in a SPARC promoter occurring in an animal, wherein the mutation results in reduced intracellular and/or extracellular levels of a polypeptide encoded by the SPARC gene, the method including: determining the nucleic acid sequence of the SPARC promoter, and comparing the determined the SPARC gene nucleic acid sequence to a database of wild-type nucleic acid sequences which correspond to the determined Oligonucleotide or peptide nucleic acid nucleic acid sequence. The method may further including obtaining nucleic acids from the animal and or amplification of the nucleic acid prior to determining the nucleic acid sequence. The presence of a mutation may be indicative of a cancer in the animal having resistance to a therapeutic regimen.

In accordance with another aspect of the invention, methods are provided for treating a cancer in a subject in need thereof, the method including administering to the subject a therapeutic regimen, wherein said subject has a SPARC genotype indicative of sensitivity to the therapeutic regimen.

In accordance with another aspect of the invention, methods are provided for treating a cancer in a subject in need thereof, the method including: selecting a subject having a SPARC promoter genotype indicative of sensitivity to a therapeutic regimen; and administering to said subject the therapeutic regimen.

In accordance with another aspect of the invention, methods are provided for treating a cancer in a subject in need thereof, the method including: selecting a subject having a resistant genotype in a SPARC promoter sequence; and administering to said subject an alternative therapeutic regimen.

In accordance with another aspect of the invention, methods are provided for identifying a subject with an increased sensitivity to a therapeutic regimen for treating a cancer in a subject in need thereof, including the step of screening a population of subjects to identify those subjects that have a mutation in their SPARC promoter sequence, wherein the identification of a subject with a mutation is predictive of decreased responsiveness to the therapeutic regimen.

In accordance with another aspect of the invention, methods are provided for treating a cancer in a subject in need thereof, the method including administering a therapeutic regimen to the subject, wherein said subject does not have a mutation indicative of resistance to the therapeutic regimen in their SPARC promoter sequence.

The one or more SPARC promoter mutations, may be selected from one or more of the following mutations relative to SEQ ID NO: 1: (a) 295delG and 301A>CC; (b) 315insA and 316G>A; (c) 341C>T, 342T>C, 344A>C, 347C>A, 354G>A, 356G>A, 359T>G and 363G>A; (d) 395C>T and 396A>T; (e) 488T>C; (f) 533A>G; (g) 734G>A; (h) 734delG; (i) 769T>A, 771G>C and 774G>C; (j) 784T>C, 786T>G and 788T>A; (k) 793T>G, 796T>A and 800T>C; (l) 820C>A and 824T>A; (m) 864T>A and 867T>A; (n) 895T>G, 899T>A, 901G>A and 904G>A; (o) 901G>A; (p) 901G>A, 904G>A, 929A>G and 934delC; (q) 922T>G, 928T>G and 935T>G; (r) 982T>C, 984T>G, 985G>A, 991G>A, 995G>A, 999C>T and 1000T>G; (s) 1063G>C; (t) 1079G>A; (u) 1085delC, 1086delA, 1o 1087delG, 1089G>A, 1090T>C, 1091T>C and 1092G>T; (v) 1161C>A, 1162T>C, 1163delA and 1164delT; and (w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T. The mutation result in reduced intracellular and/or extracellular levels of a polypeptide encoded by the SPARC promoter sequence. The therapeutic regimen may be selected from: radiotherapy, a first line chemotherapy or a combination thereof. The therapeutic regimen may include the administration of one or more chemotherapeutic agents. The chemotherapeutic agent may be selected from 5-flurouracil, irinotecan, cisplatin, doxorubicin or combinations thereof.

The one or more SPARC promoter mutations may be indicative of the cancer's resistance to the therapeutic regimen. The method may further include obtaining SPARC promoter information for the subject. The one or more SPARC promoter mutations may be determined using a nucleic acid sample from the subject. The method may further include obtaining a nucleic acid sample from the subject. The method obtaining the nucleic acid sample may be from a biological sample taken from the subject's cancer. The biological sample may be obtained from a biopsy. The biological samples may also be repeatedly obtained at various stages during the therapeutic regimen.

Determining the presence or an absence of one or more SPARC promoter mutations in the subject's cancer may be done using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and/or reading sequence data.

The cancer may be selected from one or more of the following: carcinoma; melanoma; sarcoma; lymphoma; and leukaemia. The carcinoma may be selected from the following: squamous cell; adeno; small cell; large cell and combinations thereof. The carcinoma may be derived from a primary tumor located in nasopharygeal tissue, head and neck, lung, esophagus, stomach, intestine, rectum, kidney, urinary tract, prostate, testes, ovary, uterus, cervix, vagina, skin, and endocrine glands. The sarcoma may be selected from the following tissues: muscle; adipose; fibrous; vascular; and neural. The lymphoma may be selected from the following: Hodgkin's Disease; and Non-Hodgkins Lymphoma. The leukaemia may be selected from the following: lymphoid; and myeloid. The cancer may be a colorectal carcinoma, prostate carcinoma, ovarian carcinoma or osteosarcoma. The cancer may be a SPARC inhibited cancer. The SPARC inhibited cancer may be a colorectal carcinoma, prostate carcinoma, ovarian carcinoma, osteosarcoma or uterine sarcoma.

### DETAILED DESCRIPTION OF THE INVENTION

### 1. Definitions

In the description that follows, a number of terms are used extensively, the following definitions are provided to facilitate understanding of the invention.

A “nucleic acid sample” as used herein includes any nucleic acid, and may be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). “Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′ 5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the mutation of interest. Numerous examples of SPARC gene sequences, both wild type and mutated, are described herein and provided by SEQ ID NO: 1-55.

A “genotype” is defined as the genetic constitution of an organism, usually in respect to one gene or a few genes or a region of a gene relevant to a particular context (i.e. the genetic loci responsible for a particular phenotype). For example, as described herein the genotype can refer to the presence or absence of a particular mutation within a SPARC promoter sequence indicative of sensitivity or resistance to a therapeutic regimen.

A “promoter” region is 5′ or upstream of the translation start site or ATG start codon.

It will be appreciated by a person of skill in the art that the numerical designations of the positions of mutations within a sequence are relative to the specific sequence. Also the same positions may be assigned different numerical designations depending on the way in which the sequence is numbered and the sequence chosen, as illustrated by the alternative numbering in HAFNER et al. (1994) supra, in which a SPARC promoter sequence, represented by accession number X82259 has numerous numbering and sequence differences compared to U65081.1 (JENDRASCHAK E. and KAMINSKI W E. (1998) supra). Furthermore, sequence variations such as insertions or deletions, may change the relative position and subsequently the numerical designations of particular nucleotides at and around a mutational site.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, as shown in SEQ ID NO:2.

A “mutation” as described herein may be the result of a “single nucleotide polymorphism” (SNP) occurring at a polymorphic site occupied by a single nucleotide, which is the site of variation between allelic sequences. A single nucleotide polymorphism may arise due to substitution of one nucleotide for another at the polymorphic site. A “transition” is the replacement of one purine by another purine or one pyrimidine by another pyrimidine. A “transversion” is the replacement of a purine by a pyrimidine or vice versa. A mutation may also arise from a deletion (represented by “−” or “del”) of one or more nucleotides or an insertion (represented by “+” or “ins”) of one or more nucleotides relative to a reference sequence or wildtype (wt) sequence (for example SEQ ID NO: 1). Alternatively, a mutation may result in a frameshift of the sequence resulting from a deletion or insertion as described above or from an inversion etc. Additionally, a mutation as described herein may be a multisite mutation, whereby the mutation is comprised of two or more mutations. Examples of multisite mutations are found as described in (a)-(d), (i)-(n), (p)-(r) and (u)-(w). Specifically, (b) is a combination of 315insA and 316G>A relative to SEQ ID NO: 1. Furthermore, it would be appreciated by a person of skill in the art, that an insertion or deletion within a given sequence could alter the relative position and therefore the position number of another mutation within the sequence.

SPARC promoter mutations relative to SEQ ID NO: 1 are shown in TABLE 1 below.

TABLE 1

| Mutation | |
|---|---|
| Identifier | SPARC MUTATIONS RELATIVE TO SEQ ID NO: 1 |
| (a) | 295delG and 301A > CC |
| (b) | 315insA and 316G > A |
| (c) | 341C > T, 342T > C, 344A > C, 347C > A, 354G > A, |
| | 356G > A, 359T > G and 363G > A |
| (d) | 395C > T and 396A > T |
| (e) | 488T > C |
| (f) | 533A > G |
| (g) | 734G > A |
| (h) | 734delG |
| (i) | 769T > A, 771G > C and 774G > C |
| (j) | 784T > C, 786T > G and 788T > A |
| (k) | 793T > G, 796T > A and 800T > C |
| (l) | 820C > A and 824T > A |
| (m) | 864T > A and 867T > A |
| (n) | 895T > G, 899T > A, 901G > A and 904G > A |
| (o) | 901G > A |
| (p) | 901G > A, 904G > A, 929A > G and 934delC |
| (q) | 922T > G, 928T > G and 935T > G |
| (r) | 982T > C, 984T > G, 985G > A, 991G > A, 995G > A, |
| | 999C > T and 1000T > G |
| (s) | 1063G > C |
| (t) | 1079G > A |
| (u) | 1085delC, 1086delA, 1087delG, 1089G > A, 1090T > C, |
| | 1091T > C and 1092G > T |
| (v) | 1161C > A, 1162T > C, 1163delA and 1164delT |
| (w) | 1215C > T, 1216delG, 1217delG, 1218delG, 1219delG, |
| | 1220delT, 1221delG, 1222delG, 1223delA, 1224delG, |
| | 1225delG, 1226delG, 1227delG, 1228delA, 1229delG, |
| | 1230delA, 1231delT, 1232delA, 1233delG, 1234delA, |
| | 1235delC, 1236delC and 1237C > T |

A “polymorphic site” or “polymorphism site” or “polymorphism” or “single nucleotide polymorphism site” (SNP site) as used herein is the locus or position with in a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, enhancers and introns) of genes.

A “SPARC promoter mutation” as used herein refers to any mutation which may be found in a SPARC promoter sequence (for example, SEQ ID NOs: 1-11, but also Accession No. X82259). Such SPARC mutations may be indicative of sensitivity to a therapeutic regimen directed at a cancer whose nucleic acids contain the mutation. For example, SPARC mutations are listed in TABLE 1 ((a)-(w) relative to SEQ ID NO: 1) supra and in SEQ ID NOS:2-33.

A “therapeutic regimen” as described herein may be a chemotherapeutic regimen or a radiotherapy regimen or a combination thereof.

As used herein, a “chemotherapeutic regimen” or “chemotherapy” refers to the use of at least one first line chemotherapy agent to destroy cancerous cells, including leukemia and lymphoma. There are a myriad of such first line chemotherapy agents available to a clinician. Chemotherapy agents may be administered to a subject in a single bolus dose, or may be administered in smaller doses over time. A single chemotherapeutic agent may be used (single-agent therapy) or more than one agent may be used in combination (combination therapy). Chemotherapy may be used alone to treat some types of cancer. Alternatively, chemotherapy may be used in combination with other types of treatment, for example, radiotherapy or “alternative therapies (for example immunotherapy) as described herein.

As used herein, a first line “chemotherapeutic agent” or first line chemotherapy is a medicament that may be used to treat cancer, and generally has the ability to kill cancerous cells directly. Examples of first line chemotherapeutic agents include alkylating agents, antimetabolites, natural products, hormones and antagonists, and miscellaneous agents. Examples of alternate names are indicated in brackets. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib. Alternate names and trade-names of these and additional examples of chemotherapeutic agents, and their methods of use including dosing and administration regimens, will be known to a physician versed in the art, and may be found in, for example “The Pharmacological basis of therapeutics”, 10^{th} edition. HARDMAN H G., LIMBIRD L E. editors. McGraw-Hill, New York, and in “Clinical Oncology”, 3^{rd} edition. Churchill Livingstone/Elsevier Press, 2004. ABELOFF, M D. editor.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

Whether direct or indirect damage from radiotherapy results in the loss of cell division and therefore cell growth. Accordingly, many radiation damaged cells are only detected when they try to undergo cell division and associated processes (for example replication). However, some cells will actually make it through a few rounds of cell division before the damage results in cell death, while others may be recognized a damaged sooner and undergo apoptosis before the cell even attempts division. Therefore depending on the type of radiation applied, the intensity of the radiation, the cell type and other factors, it may be apparent that a cell has been damaged by a radiotherapeutic regimen or it may not, even with histologic review.

Radiotherapy may further be used in combination chemotherapy, with the chemotherapeutic agent acting as a radiosensitizer. The specific choice of radiotherapy suited to an individual patient may be determined by a physician or oncologist, taking into consideration the tissue and stage of the cancer. Examples of radiotherapy approaches to various cancers may be found in, for example “Clinical Oncology”, 3^{rd} edition. Churchill Livingstone/Elsevier Press, 2004. ABELOFF, M D. editor.

As used herein the term “alternative therapeutic regimen” or “alternative therapy” (not a first line chemotherapeutic regimen as described above) may include for example, SPARC polypeptide (as described in WO 2004/064785), receptor tyrosine kinase inhibitors (for example Iressa™ (gefitinib), Tarceva™ (erlotinib), Erbitux™ (cetuximab), imatinib mesilate (Gleevec™), proteosome inhibitors (for example bortezomib, Velcade™); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, Rapamune™); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rouse sarcoma virus transforming oncogene (SRC) inhibitors, histonedeacetylase inhibitors, small hypoxia-inducible factor inhibitors, hedgehog inhibitors, and TGF-β signalling inhibitors. Furthermore, an immunotherapeutic agent would also be considered an alternative therapeutic regimen. For example, serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Alternate names and trade-names of these agents used in alternative therapeutic regimens and additional examples of agents used in alternative therapeutic regimens, and their methods of use including dosing and administration regimens, will be known to a physician versed in the art. Furthermore, methods of use of chemotherapeutic agents and other agents used in alternative therapeutic regimens in combination therapies, including dosing and administration regimens, will also be known to a physician versed in the art.

The detection of nucleic acids in a sample may rely on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High stringency hybridization is relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in AUSUBEL et al., Current Protocols in Molecular Biology, John Wiley & Sons, New York, N.Y., 1998.

A representative of a Homo sapiens SPARC promoter gene sequence is listed in GenBank under accession number U65081 (SEQ ID NO: 1). Polymorphic sites in SPARC promoter sequence are identified by their variant (i.e. M, W, Y, S, R, K or V) in SEQ ID NO:2.

TABLE 2

Primers

| SEQ | Primer | | |
|---|---|---|---|
| ID | Sequence | Nucleotide Position | Primer name |
| 34 | TTCTGCACTT | 35-57 of SEQ ID NO: 9 | Set 1 fwd |
| | AACTCGTCTA | | |
| | AA | | |
| 35 | GCTGATTCTA | 546-567 of SEQ ID NO: 9 | Set 1 rev |
| | CGTTTCAACT | (on opposite strand) | |
| | TG | | |
| 36 | TGAGGACAAG | 431-452 of SEQ ID NO: 9 | Set 2 fwd |
| | GACCAGGTAT | | |
| | TT | | |
| 37 | AGGAAACCAC | 1360-1380 of SEQ ID | Set 2 rev |
| | TCAGAGCTC NO: 9 (on opposite | | |
| | TG | strand) | |
| 38 | GTCATCCCTC | 1126-1141 of SEQ ID | Set 3 fwd |
| | CAGGCC | NO:9 | |
| 39 | CGGGGTTGGT | 2905-2924 of SEQ ID | Set 3 rev |
| | GCAACTATA | NO: 9 (on opposite | |
| | | strand) | |
| 40 | Tgtaaaacga | 430-452 of SEQ ID NO: | SPARC fwd |
| | cggccagTT | 9 | (with 5′ M13 |
| | GAGGACAAG | | tag in lower |
| | GACCAGGTA | | case) |
| | TTT | | |
| 41 | Caggaaacag | 1359-1379 of SEQ ID | SPARC rev |
| | ctatgacAGG | NO: 9 (on opposite | (with 5′ M13 |
| | AAACCACTCA | strand) | tag in lower |
| | GAGCTCTG | | case) |
| 42 | Tgtaaaacga | 1338-1354 of SEQ ID | sp-seq-1-F |
| | cggccagTGG | NO: 9 | (with 5′ M13 |
| | TGGAGGGGAG | | tag in lower |
| | ATAGACC | | case) |
| 43 | Caggaaacag | 2140-2158 of X82259 | sp seq 1-R |
| | ctatgacTCC | (on opposite strand) | (with M13 tag |
| | CAAGGAACTC | | in lower case) |
| | AGGACAG | | |
| 44 | Tgtaaaacga | 430-452 of SEQ ID NO: 9 | sp seq-up-1-F |
| | cggccagTTG | | (with 5′ M13 |
| | AGGACAAGGA | | tag in lower |
| | CCAGGTATTT | | case) |
| 45 | Caggaaacag | 1360 -1380 of SEQ ID | sp seq-up-1-R |
| | ctatgacAGG | NO: 9 (on opposite | (with 5′ M13 |
| | AAACCACTCA | strand) | tag in lower |
| | GAGCTCTG | | case) |
| 46 | Tgtaaaacga | 2097-2116 of X82259 | sp seq 2-F |
| | cggccagTAA | | (with 5′ M13 |
| | ATGGCACCAT | | tag in lower |
| | CACAACCT | | case) |
| 47 | Caggaaacag | Intronic | sp seq 2-R |
| | ctatgacCCG | | (with 5′ M13 |
| | AAGCCTGTGT | | tag in lower |
| | CCTACTT | | case) |
| 48 | Tgtaaaacga | 31-52 of X82259 | sp-seq up-0-F |
| | cggccagTTT | | (with 5′ M13 |
| | CTGCACTTAA | | tag in lower |
| | CTCGTCTAAA | | case) |
| 49 | Caggaaacag | 546-567 of SEQ ID NO: | sp-seq up-0-R |
| | ctatgacgCT | 9 (on opposite strand) | (with 5′ M13 |
| | GATTCTACGT | | tag in lower |
| | TTCAACTTG | | case) |
| 50 | Tgtaaaacga | 1126-1141 of SEQ ID | sp-seq up-2-F |
| | cggccagtGT | NO:9 | (with 5′ M13 |
| | CATCCCTCCA | | tag in lower |
| | GGCC | | case) |
| 51 | Caggaaacag | 1527-1512 of X82259 | sp-seq up-2-R |
| | ctatgacCGG | | (with 5′ M13 |
| | GGTTGGTGCA | | tag in lower |
| | ACTATA | | case) |
| 52 | CGAAGAGGAG | (1553-1575 of SEQ ID | SPARC fwd |
| | GTGGTGGCG | NO: 1) | (TAI 2005) |
| | GAAA | | |
| 53 | GGTTGTTGTC | 1901-1926 of SEQ ID | SPARC rev |
| | CTCATCCCTC | NO: 1 (opposite strand) | (TAI 2005) |
| | TCATAC | | |
| 54 | CTCTCTGCTC | Not SPARC | GAPDH fwd |
| | CTCCTGTTCG | | (TAI 2005) |
| | ACAG | | |
| 55 | AGGGGTCTTA | Not SPARC | GAPDH rev |
| | CTCCTTGGAG | | (TAI 2005) |
| | GCCA | | |

### Sparc Promoter Sequencing Primers

Sequencing primers corresponding to SEQ ID NO: 34 and SEQ ID NO: 35 were used to obtain sequence information on both strands of the SPARC promoter (SEQ ID NO: 1) for the region between nucleotide 1 and nucleotide 425.

Sequencing primers corresponding to SEQ ID NO: 36 and SEQ ID NO: 37 were used to obtain sequence information on both strands of the SPARC promoter (SEQ ID NO: 1) for the region between nucleotide 333 and nucleotide 1241.

Sequencing primers corresponding to SEQ ID NO: 38 and SEQ ID NO: 39 were used to obtain sequence information on both strands of the SPARC promoter (SEQ ID NO: 1) for the region between nucleotide 1024 and nucleotide 1370.

The sequences shown in TABLE 3 below (SEQ ID NOS: 11-33) show SPARC promoter mutations (underligned) with flanking sequences. The numbering associated with these sequences represents the mutation positions relative to SEQ ID NO:1 (wild type SPARC promoter sequence).

TABLE 3

| SPARC | SEQ | |
|---|---|---|
| PROMOTER | ID | |
| Mutations | NO: | MUTATION(S) WITH FLANKING SEQUENCE |
| 295delG and | 11 | AATAAATTAGCTGGGTGTTGTGGCATGTGCGCCT |
| 301A > CC | | GTAATCCCAGCTACTCTGGAGGCTGAGGCGCGAT |
| | | AATTGCTTGAACCCGGGAGGCAGAGGTTGCAGTG |
| | | AGCCGAAATCATACCACTGCACTCCAGCCTGGGC |
| | | GACAGAGTGAGTGAGACTCTGTCTCAAAACAAAA |
| | | CAAAACAAACAAACAAAAAAACCG_AAACCCCCA |
| | | AAACTTTTTGAGGACAAGGACCAGATATTTATTA |
| | | ATTCTCATACCTCCCAGAGTGTTAGGCACGAAAT |
| | | AAACATTCAACCAAGACCTGTTGCACTGAGCAGT |
| | | TCATATATAACAGGAGTGACCCAAGTTGAAACGT |
| | | AGAATCAGCCCTCTCATACCACTTTTTGGCAGGT |
| | | GATCATAGGCAAGTTACTTAGCATCT |
| 315insA and | 12 | GTGTTGTGGCATGTGCGCCTGTAATCCCAGCTAC |
| 316G > A | | TCTGGAGGCTGAGGCGCGATAATTGCTTGAACCC |
| | | GGGAGGCAGAGGTTGCAGTGAGCCGAAATCATAC |
| | | CACTGCACTCCAGCCTGGGCGACAGAGTGAGTGA |
| | | GACTCTGTCTCAAAACAAAACAAAACAAACAAAC |
| | | AAAAAAACCGGAAACCACAAAACTTTTTGAAGA A |
| | | CAAGGACCAGATATTTATTAATTCTCATACCTCC |
| | | CAGAGTGTTAGGCACGAAATAAACATTCAACCAA |
| | | GACCTGTTGCACTGAGCAGTTCATATATAACAGG |
| | | AGTGACCCAAGTTGAAACGTAGAATCAGCCCTCT |
| | | CATACCACTTTTTGGCAGGTGATCATAGGCAAGT |
| | | TACTTAGCATCTATGTTTCCTTATTATTAAAAT |
| 341C > T, | 13 | CCAGCTACTCTGGAGGCTGAGGCGCGATAATTGC |
| 342T > C, | | TTGAACCCGGGAGGCAGAGGTTGCAGTGAGCCGA |
| 344A > C, | | AATCATACCACTGCACTCCAGCCTGGGCGACAGA |
| 347C > A, | | GTGAGTGAGACTCTGTCTCAAAACAAAACAAAAC |
| 354G > A, | | AAACAAACAAAAAAACCGGAAACCACAAAACTTT |
| 356G > A, | | TTGAGGACAAGGACCAGATATTTATTAATTTCCC |
| 359T > G and | | CTAACTCCCAAAATGGTAGaCACGAAATAAACAT |
| 363G > A | | TCAACCAAGACCTGTTGCACTGAGCAGTTCATAT |
| | | ATAACAGGAGTGACCCAAGTTGAAACGTAGAATC |
| | | AGCCCTCTCATACCACTTTTTGGCAGGTGATCAT |
| | | AGGCAAGTTACTTAGCATCTATGTTTCCTTATTA |
| | | TTAAAATGGTCATAATTACAATGCCTA |
| 395C > T and | 14 | TTGCAGTGAGCCGAAATCATACCACTGCACTCCA |
| 396A > T | | GCCTGGGCGACAGAGTGAGTGAGACTCTGTCTCA |
| | | AAACAAAACAAAACAAACAAACAAAAAAACCGGA |
| | | AACCACAAAACTTTTTGAGGACAAGGACCAGATA |
| | | TTTATTAATTCTCATACCTCCCAGAGTGTTAGGC |
| | | ACGAAATAAACATTCAACCAAGACCTGTTGTTCT |
| | | GAGCAGTTCATATATAACAGGAGTGACCCAAGTT |
| | | GAAACGTAGAATCAGCCCTCTCATACCACTTTTT |
| | | GGCAGGTGATCATAGGCAAGTTACTTAGCATCTA |
| | | TGTTTCCTTATTATTAAAATGGTCATAATTACAA |
| | | TGCCTAAGATAAGGGGTTGCTGTGAAGATTATTA |
| | | AATCCTCAGTAAACTTTGGCTATTGTTACTCC |
| 488T > C | 15 | AAAACCGGAAACCACAAAACTTTTTGAGGACAAG |
| | | GACCAGATATTTATTAATTCTCATACCTCCCAGA |
| | | GTGTTAGGCACGAAATAAACATTCAACCAAGACC |
| | | TGTTGCACTGAGCAGTTCATATATAACAGGAGTG |
| | | ACCCAAGTTGAAACGTAGAATCAGCCCTCTCATA |
| | | CCACTTTTTGGCAGGTGATCATAGGCAAGTCACT |
| | | TAGCATCTATGTTTCCTTATTATTAAAATGGTCA |
| | | TAATTACAATGCCTAAGATAAGGGGTTGCTGTGA |
| | | AGATTATTAAATCCTCAGTAAACTTTGGCTATTG |
| | | TTACTCCTATGATTATCATCAATATCATCACTTA |
| | | CCTTATCTGTTCAATACTGGTGGCACAGGTCCAC |
| | | CAGCTAGATGTCTAATCCCTTATGTGTCT |
| 533A > G | 16 | TATTAATTCTCATACCTCCCAGAGTGTTAGGCAC |
| | | GAAATAAACATTCAACCAAGACCTGTTGCACTGA |
| | | GCAGTTCATATATAACAGGAGTGACCCAAGTTGA |
| | | AACGTAGAATCAGCCCTCTCATACCACTTTTTGG |
| | | CAGGTGATCATAGGCAAGTTACTTAGCATCTATG |
| | | TTTCCTTATTATTAAAATGGTCATAATTACGATG |
| | | CCTAAGATAAGGGGTTGCTGTGAAGATTATTAAA |
| | | TCCTCAGTAAACTTTGGCTATTGTTACTCCTATG |
| | | ATTATCATCAATATCATCACTTACCTTATCTGTT |
| | | CAATACTGGTGGCACAGGTCCACCAGCTAGATGT |
| | | CTAATCCCTTATGTGTCTATTAGTGGTACAAGTG |
| | | GAGTTTGAGTGGGATTTTTTTTTTTAAGACCAGT |
| 734G > A | 17 | ATGCCTAAGATAAGGGGTTGCTGTGAAGATTATT |
| | | AAATCCTCAGTAAACTTTGGCTATTGTTACTCCT |
| | | ATGATTATCATCAATATCATCACTTACCTTATCT |
| | | GTTCAATACTGGTGGCACAGGTCCACCAGCTAGA |
| | | TGTCTAATCCCTTATGTGTCTATTAGTGGTACAA |
| | | GTGGAGTTTGAGTGGGATTTTTTTTTTTAAAACC |
| | | AGTTCCAAATCATCAAGGATGATACCACTAGTAG |
| | | CAGCTTGTCTTGTCTGTACAGTGGTAAGTCCTGG |
| | | CCTTGCCTTTGTGGCAAATACAACCCCCTTGAAT |
| | | TGCTTGGCCCTTCTCAGCATTGCCTAATATTAGG |
| | | GAGGACTCCTGTAAAGCTCACTGGTTAGAAGATC |
| | | AAGACACTTGGGCCTGGTTCTACCCCCTGGGGG |
| 734delG | 18 | ATGCCTAAGATAAGGGGTTGCTGTGAAGATTATT |
| | | AAATCCTCAGTAAACTTTGGCTATTGTTACTCCT |
| | | ATGATTATCATCAATATCATCACTTACCTTATCT |
| | | GTTCAATACTGGTGGCACAGGTCCACCAGCTAGA |
| | | TGTCTAATCCCTTATGTGTCTATTAGTGGTACAA |
| | | GTGGAGTTTGAGTGGGATTTTTTTTTTTAA_ACC |
| | | AGTTCCAAATCATCAAGGATGATACCACTAGTAG |
| | | CAGCTTGTCTTGTCTGTACAGTGGTAAGTCCTGG |
| | | CCTTGCCTTTGTGGCAAATACAACCCCCTTGAAT |
| | | TGCTTGGCCCTTCTCAGCATTGCCTAATATTAGG |
| | | GAGGACTCCTGTAAAGCTCACTGGTTAGAAGATC |
| | | AAGACACTTGGGCCTGGTTCTACCCCCTGGGGG |
| 769T > A, | 19 | AATCCTCAGTAAACTTTGGCTATTGTTACTCCTA |
| 771G > C and | | TGATTATCATCAATATCATCACTTACCTTATCTG |
| 774G > C | | TTCAATACTGGTGGCACAGGTCCACCAGCTAGAT |
| | | GTCTAATCCCTTATGTGTCTATTAGTGGTACAAG |
| | | TGGAGTTTGAGTGGGATTTTTTTTTTTAAGACCA |
| | | GTTCCAAATCATCAAGGATGATACCACTAGAACC |
| | | ACCTTGTCTTGTCTGTACAGTGGTAAGTCCTGGC |
| | | CTTGCCTTTGTGGCAAATACAACCCCCTTGAATT |
| | | GCTTGGCCCTTCTCAGCATTGCCTAATATTAGGG |
| | | AGGACTCCTGTAAAGCTCACTGGTTAGAAGATCA |
| | | AGACACTTGGGCCTGGTTCTACCCCCTGGGGGCC |
| | | ATTGGGTAATTCCTTGCAGTCTCCAGGC |
| 784T > C, | 20 | TTGGCTATTGTTACTCCTATGATTATCATCAATA |
| 786T > G and | | TCATCACTTACCTTATCTGTTCAATACTGGTGGC |
| 788T > Z | | ACAGGTCCACCAGCTAGATGTCTAATCCCTTATG |
| | | TGTCTATTAGTGGTACAAGTGGAGTTTGAGTGGG |
| | | ATTTTTTTTTTTAAGACCAGTTCCAAATCATCAA |
| | | GGATGATACCACTAGTAGCAGCTTGTCTTGCCGG |
| | | AACAGTGGTAAGTCCTGGCCTTGCCTTTGTGGCA |
| | | AATACAACCCCCTTGAATTGCTTGGCCCTTCTCA |
| | | GCATTGCCTAATATTAGGGAGGACTCCTGTAAAG |
| | | CTCACTGGTTAGAAGATCAAGACACTTGGGCCTG |
| | | GTTCTACCCCCTGGGGGCCATTGGGTAATTCCTT |
| | | GCAGTCTCCAGGCCTCACTTGCCCTCTGAACAA |
| 793T > G, | 21 | GTTACTCCTATGATTATCATCAATATCATCACTT |
| 796T > A and | | ACCTTATCTGTTCAATACTGGTGGCACAGGTCCA |
| 800T > C | | CCAGCTAGATGTCTAATCCCTTATGTGTCTATTA |
| | | GTGGTACAAGTGGAGTTTGAGTGGGATTTTTTTT |
| | | TTTAAGACCAGTTCCAAATCATCAAGGATGATAC |
| | | CACTAGTAGCAGCTTGTCTTGTCTGTACAGGGGA |
| | | AAGCCCTGGCCTTGCCTTTGTGGCAAATACAACC |
| | | CCCTTGAATTGCTTGGCCCTTCTCAGCATTGCCT |
| | | AATATTAGGGAGGACTCCTGTAAAGCTCACTGGT |
| | | TAGAAGATCAAGACACTTGGGCCTGGTTCTACCC |
| | | CCTGGGGGCCATTGGGTAATTCCTTGCAGTCTCC |
| | | AGGCCTCACTTGCCCTCTGAACAA |
| 820C > A and | 22 | ATCACTTACCTTATCTGTTCAATACTGGTGGCAC |
| 824T > A | | AGGTCCACCAGCTAGATGTCTAATCCCTTATGTG |
| | | TCTATTAGTGGTACAAGTGGAGTTTGAGTGGGAT |
| | | TTTTTTTTTTAAGACCAGTTCCAAATCATCAAGG |
| | | ATGATACCACTAGTAGCAGCTTGTCTTGTCTGTA |
| | | CAGTGGTAAGTCCTGGCCTTGCCTTTGTGGAAAA |
| | | AACAACCCCCTTGAATTGCTTGGCCCTTCTCAGC |
| | | ATTGCCTAATATTAGGGAGGACTCCTGTAAAGCT |
| | | CACTGGTTAGAAGATCAAGACACTTGGGCCTGGT |
| | | TCTACCCCCTGGGGGCCATTGGGTAATTCCTTGC |
| | | AGTCTCCAGGCCTCACTTGCCCTCTGAACAAGAA |
| | | AGAGGCTGTTCTGGGTCATCCCTCCAG |
| 864T > A and | 23 | GCTAGATGTCTAATCCCTTATGTGTCTATTAGTG |
| 867T > A | | GTACAAGTGGAGTTTGAGTGGGATTTTTTTTTTT |
| | | AAGACCAGTTCCAAATCATCAAGGATGATACCAC |
| | | TAGTAGCAGCTTGTCTTGTCTGTACAGTGGTAAG |
| | | TCCTGGCCTTGCCTTTGTGGCAAATACAACCCCC |
| | | TTGAATTGCTTCGCCCTTCTCAGCATTGCCAAAA |
| | | ATTAGGGAGGACTCCTGTAAAGCTCACTGGTTAG |
| | | AAGATCAAGACACTTGGGCCTGGTTCTACCCCCT |
| | | GGGGGCCATTGGGTAATTCCTTGCAGTCTCCAGG |
| | | CCTCACTTGCCCTCTGAACAAGAAAGAGGCTGTT |
| | | CTGGGTCATCCCTCCAGGCCTGTCCAGCCCTGGC |
| | | ACTCTGTGAGTCGGTTTAGGCAG |
| 895T > G, | 24 | GTGGTACAAGTGGAGTTTGAGTGGGATTTTTTTT |
| 899T > A, | | TTTAAGACCAGTTCCAAATCATCAAGGATGATAC |
| 901G > A and | | CACTAGTAGCAGCTTGTCTTGTCTGTACAGTGGT |
| 904G > A | | AAGTCCTGGCCTTGCCTTTGTGGCAAATACAACC |
| | | CCCTTGAATTGCTTGGCCCTTCTCAGCATTGCCT |
| | | AATATTAGGGAGGACTCCTGTAAAGCTCACGGGT |
| | | AAAAAAATCAAGACACTTGGGCCTGGTTCTACCC |
| | | CCTGGGGGCCATTGGGTAATTCCTTGCAGTCTCC |
| | | AGGCCTCACTTGCCCTCTGAACAAGAAAGAGGCT |
| | | GTTCTGGGTCATCCCTCCAGGCCTGTCCAGCCCT |
| | | GGCACTCTGTGAGTCGGTTTAGGCAGCAGCCCCG |
| | | GAACAGATGAGGCAGGCAGGGTTGGGACGTTTGG |
| | | TCAGGACAGCCCACCGCACCAAGAGGAGGAAAGA |
| | | AATG |
| 901G > A | 25 | CAAGTGGAGTTTGAGTGGGATTTTTTTTTTTAAG |
| | | ACCAGTTCCAAATCATCAAGGATGATACCACTAG |
| | | TAGCAGCTTGTCTTGTCTGTACAGTGGTAAGTCC |
| | | TGGCCTTGCCTTTGTGGCAAATACAACCCCCTTG |
| | | AATTGCTTGGCCCTTCTCAGCATTGCCTAATATT |
| | | AGGGAGGACTCCTGTAAAGCTCACTGGTTAAAAG |
| | | ATCAAGACACTTGGGCCTGGTTCTACCCCCTGGG |
| | | GGCCATTGGGTAATTCCTTGCAGTCTCCAGGCCT |
| | | CACTTGCCCTCTGAACAAGAAAGAGGCTGTTCTG |
| | | GGTCATCCCTCCAGGCCTGTCCAGCCCTGGCACT |
| | | CTGTGAGTCGGTTTAGGCAGCAGCCCCGGAACAG |
| | | ATGAGGCAGGCAGGGTTGGGACGTTTGGTCAGGA |
| | | CAGCCCACCGCACCAAGAGGAGGAAAGAAATG |
| 901G > A, | 26 | CAAGTGGAGTTTGAGTGGGATTTTTTTTTTTAAG |
| 904G > A, | | ACCAGTTCCAAATCATCAAGGATGATACCACTAG |
| 929A > G and | | TAGCAGCTTGTCTTGTCTGTACAGTGGTAAGTCC |
| 934delC | | TGGCCTTGCCTTTGTGGCAAATACAACCCCCTTG |
| | | AATTGCTTGGCCCTTCTCAGCATTGCCTAATATT |
| | | AGGGAGGACTCCTGTAAAGCTCACTGGTTAAAAA |
| | | ATCAAGACACTTGGGCCTGGTTCTGCCCC_TGGG |
| | | GGCCATTGGGTAATTCCTTGCAGTCTCCAGGCCT |
| | | CACTTGCCCTCTGAACAAGAAAGAGGCTGTTCTG |
| | | GGTCATCCCTCCAGGCCTGTCCAGCCCTGGCACT |
| | | CTGTGAGTCGGTTTAGGCAGCAGCCCCGGAACAG |
| | | ATGAGGCAGGCAGGGTTGGGACGTTTGGTCAGGA |
| | | CAGCCCACCGCACCAAGAGGAGGAAAGAAATG |
| 922T > G, | 27 | TTTTTTTTTTAAGACCAGTTCCAAATCATCAAGG |
| 928T > G and | | ATGATACCACTAGTAGCAGCTTGTCTTGTCTGTA |
| 935T > G | | CAGTGGTAAGTCCTGGCCTTGCCTTTGTGGCAAA |
| | | TACAACCCCCTTGAATTGCTTGGCCCTTCTCAGC |
| | | ATTGCCTAATATTAGGGAGGACTCCTGTAAAGCT |
| | | CACTGGTTAGAAGATCAAGACACTTGGGCCGGGT |
| | | TCGACCCCCGGGGGGCCATTGGGTAATTCCTTGC |
| | | AGTCTCCAGGCCTCACTTGCCCTCTGAACAAGAA |
| | | AGAGGCTGTTCTGGGTCATCCCTCCAGGCCTGTC |
| | | CAGCCCTGGCACTCTGTGAGTCGGTTTAGGCAGC |
| | | AGCCCCGGAACAGATGAGGCAGGCAGGGTTGGGA |
| | | CGTTTGGTCAGGACAGCCCACCGCACCAAGAGGA |
| | | GGAAAGAAATG |
| 982T > C, | 28 | TGTCTGTACAGTGGTAAGTCCTGGCCTTGCCTTT |
| 984T > G, | | GTGGCAAATACAACCCCCTTGAATTGCTTGGCCC |
| 985G > A, | | TTCTCAGCATTGCCTAATATTAGGGAGGACTCCT |
| 991G > A, | | GTAAAGCTCACTGGTTAGAAGATCAAGACACTTG |
| 995G > A, | | GGCCTGGTTCTACCCCCTGGGGGCCATTGGGTAA |
| 999C > T and | | TTCCTTGCAGTCTCCAGGCCTCACTTGCCCCCGA |
| 1000T > G | | AACAAAAAAAAGGTGGTTCTGGGTCATCCCTCCA |
| | | GGCCTGTCCAGCCCTGGCACTCTGTGAGTCGGTT |
| | | TAGGCAGCAGCCCCGGAACAGATGAGGCAGGCAG |
| | | GGTTGGGACGTTTGGTCAGGACAGCCCACCGCAC |
| | | CAAGAGGAGGAAAGAAATGAAAGACAGAGACAGC |
| | | TTTGGCTATGGGAGAAGGAGGAGGCCGGGGGAAG |
| | | GAGGAGACAGG |
| 1063G > C | 29 | CTAATATTAGGGAGGACTCCTGTAAAGCTCACTG |
| | | GTTAGAAGATCAAGACACTTGGGCCTGGTTCTAC |
| | | CCCCTGGGGGCCATTGGGTAATTCCTTGCAGTCT |
| | | CCAGGCCTCACTTGCCCTCTGAACAAGAAAGAGG |
| | | CTGTTCTGGGTCATCCCTCCAGGCCTGTCCAGCC |
| | | CTGGCACTCTGTGAGTCGGTTTAGGCAGCACCCC |
| | | CGGAACAGATGAGGCAGGCAGGGTTGGGACGTTT |
| | | GGTCAGGACAGCCCACCGCACCAAGAGGAGGAAA |
| | | GAAATGAAAGACAGAGACAGCTTTGGCTATGGGA |
| | | GAAGGAGGAGGCCGGGGGAAGGAGGAGACAGGAG |
| | | GAGGAGGGACCACGGGGTGGAGGGGAGATAGACC |
| | | CAGCCCAGAGCTCTGAGTGGTTTCCTGTTGCCTG |
| | | TCTCTAAACC |
| 1079G > A | 30 | CTCCTGTAAAGCTCACTGGTTAGAAGATCAAGAC |
| | | ACTTGGGCCTGGTTCTACCCCCTGGGGGCCATTG |
| | | GGTAATTCCTTGCAGTCTCCAGGCCTCACTTGCC |
| | | CTCTGAACAAGAAAGAGGCTGTTCTGGGTCATCC |
| | | CTCCAGGCCTGTCCAGCCCTGGCACTCTGTGAGT |
| | | CGGTTTAGGCAGCAGCCCCGGAACAGATGAAGCA |
| | | GGCAGGGTTGGGACGTTTGGTCAGGACAGCCCAC |
| | | CGCACCAAGAGGAGGAAAGAAATGAAAGACAGAG |
| | | ACAGCTTTGGCTATGGGAGAAGGAGGAGGCCGGG |
| | | GGAAGGAGGAGACAGGAGGAGGAGGGACCACGGG |
| | | GTGGAGGGGAGATAGACCCAGCCCAGAGCTCTGA |
| | | GTGGTTTCCTGTTGCCTGTCTCTAAACC |
| 1085delC, | 31 | AAAGCTCACTGGTTAGAAGATCAAGACACTTGGG |
| 1086delA, | | CCTGGTTCTACCCCCTGGGGGCCATTGGGTAATT |
| 1087delG, | | CCTTGCAGTCTCCAGGCCTCACTTGCCCCCTGAA |
| 1089G > A, | | CAAGAAAGAGGCTGTTCTGGGTCATCCCTCCACG |
| 1090T > C, | | CCTGTCCAGCCCTGGCACTCTGTGAGTCGGTTTA |
| 1091T > C and | | GGCAGCAGCCCCGGAACAGATGAGGCAGGG A |
| 1092G > T | | CCTGGACGTTTGGTCAGGACAGCCCACCGCACCA |
| | | AGAGGAGGAAAGAAATGAAAGACAGAGACAGCTT |
| | | TGGCTATGGGAGAAGGAGGAGGCCGGGGGAAGGA |
| | | GGAGACAGGAGGAGGAGGGACCACGGGGTGGAGG |
| | | GGAGATAGACCCAGCCCAGAGCTCTGAGTGGTTT |
| | | CCTGTTGCCTGTCTCTAAACCCCTCCACATTCCC |
| | | GCGGTCCTTC |
| 1161C > A, | 32 | GTCTCCAGGCCTCACTTGCCCTCTGAACAAGAAA |
| 1162T > C, | | GAGGCTGTTCTGGGTCATCCCTCCAGGCCTGTCC |
| 1163delA and | | AGCCCTGGCACTCTGTGAGTCGGTTTAGGCAGCA |
| 1164 delT | | GCCCCGGAACAGATGAGGCAGGCAGGGTTGGGAC |
| | | GTTTGGTCAGGACAGCCCACCGCACCAAGAGGAG |
| | | GAAAGAAATGAAAGACAGAGACAGCTTTGGAC |
| | | GGGAGAAGGAGGAGGCCGGGGGAAGGAGGAGACA |
| | | GGAGGAGGAGGGACCACGGGGTGGAGGGGAGATA |
| | | GACCCAGCCCAGAGCTCTGAGTGGTTTCCTGTTG |
| | | CCTGTCTCTAAACCCCTCCACATTCCCGCGGTCC |
| | | TTCAGACTGCCCGGAGAGCGCGCTCTGCCTGCCG |
| | | CCTGCCTGCCTGCCACTGAGGGTTCCCAGCACCA |
| | | TG |
| 1215C > T, | 33 | ATCCCTCCAGGCCTGTCCAGCCCTGGCACTCTGT |
| 1216delG, | | GAGTCGGTTTAGGCAGCAGCCCCGGAACAGATGA |
| 1217delG, | | GGCAGGCAGGGTTGGGACGTTTGGTCAGGACAGC |
| 1218delG, | | CCACCGCACCAAGAGGAGGAAAGAAATGAAAGAC |
| 1219delG, | | AGAGACAGCTTTGGCTATGGGAGAAGGAGGAGGC |
| 1220delT, | | CGGGGGAAGGAGGAGACAGGAGGAGGAGGGACCA |
| 1221delG, | | T TAGCCCAGAGCT |
| 1222delG, | | CTGAGTGGTTTCCTGTTGCCTGTCTCTAAACCCC |
| 1223delA, | | TCCACATTCCCGCGGTCCTTCAGACTGCCCGGAG |
| 1224delG, | | AGCGCGCTCTGCCTGCCGCCTGCCTGCCTGCCAC |
| 1225delG, | | TGAGGGTTCCCAGCACCATG |
| 1226delG, | | |
| 1227delG, | | |
| 1228delA, | | |
| 1229delG, | | |
| 1230delA, | | |
| 1231delT, | | |
| 1232delA, | | |
| 1233delG, | | |
| 1234delA, | | |
| 1235delC, | | |
| 1236delC and | | |
| 1237C > T | | |

The Sequences given in TABLE 3 (SEQ ID NO:11-33) above and in SEQ ID NO:1-10 may be useful to a person of skill in the art in the design of further primers, and probes or other oligonucleotides or peptide nucleic acids for the identification of SPARC mutations as described herein.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. PNAS (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids.

A “vector” as used herein includes any means for delivery of a nucleic acid sample to a cell. For example, a vector may include a plasmid, artificial chromosome etc.

A “cancer” is defined as including cancers in which mutations can be detected in accordance with the methods described herein and can include but, are not limited to, carcinomas, mealomas, sarcomas, lymphomas, and leukaemias. Carcinomas may include, without limitation, squamous cell, adeno, small cell and large cell carcinomas, and combinations thereof. Carcinomas may have their primary tumor located, for example in the nasopharygeal tissue, head and neck, lung, esophagus, stomach, intestine, rectum, kidney, urinary tract, prostate, testes, ovary, uterus, cervix, vagina, skin, and endocrine glands or may be multicentric. Sarcomas may be, for example of muscle, adipose, fibrous, vascular, or neural origin. Lymphomas may include, without, limitation, for example Hodgkin's Disease and Non-Hodgkins Lymphoma. Leukaemias may be, for example, of lymphoid or myeloid cell origin and may be acute or chronic in presentation and/or course. Furthermore, a cancer may be a “SPARC inhibited cancer”, whereby the cancer decreases SPARC expression to facilitate growth of the cancer. The cancer may, for example, also be selected from colorectal carcinomas, prostate carcinomas, ovarian carcinomas, osteosarcomas or combinations thereof.

A cancer may be described as “sensitive to” or “resistant to” a given therapeutic regimen based on the ability of the regimen to kill cancer cells or decrease tumor size, reduce overall cancer growth (i.e. through reduction of angiogenesis), and or inhibit metastasis. Cancer cells that are resistant to a therapeutic regimen would not respond to the regimen and may continue to proliferate. Whereas cancer cells that are sensitive to a therapeutic regimen are those that do respond to the regimen resulting in cell death, a reduction in tumor size, reduced overall growth or inhibition of metastasis. Monitoring of a response may be accomplished by numerous pathological, clinical and imaging methods as described herein and known to persons of skill in the art.

TABLE 4
below shows correlation of SPARC mutations with values
representing resistance (R) or sensitivity (S) to a
therapeutic regimen for the treatment of cancer.

| Mutation | SPARC MUTATIONS RELATIVE TO | RESPONSE TO |
|---|---|---|
| Identifier | SEQ ID NO: 1 | THERAPY |
| (a) | 295delG and 301A > CC | R |
| (b) | 315insA and 316G > A | R |
| (c) | 341C > T, 342T > C, 344A > C, | R |
| | 347C > A, 354G > A, 356G > A, | |
| | 359T > G and 363G > A | |
| (d) | 395C > T and 396A > T | R |
| (e) | 488T > C | R |
| (f) | 533A > G | R |
| (g) | 734G > A | R |
| (h) | 734delG | R |
| (i) | 769T > A, 771G > C and 774G > C | R |
| (j) | 784T > C, 786T > G and 788T > A | R |
| (k) | 793T > G, 796T > A and 800T > C | R |
| (l) | 820C > A and 824T > A | R |
| (m) | 864T > A and 867T > A | R |
| (n) | 895T > G, 899T > A, 901G > A | R |
| | and 904G > A | |
| (o) | 901G > A | R |
| (p) | 901G > A, 904G > A, 929A > G | R |
| | and 934delC | |
| (q) | 922T > G, 928T > G and 935T > G | R |
| (r) | 982T > C, 984T > G, 985G > A, | R |
| | 991G > A, 995G > A, 999C > T | |
| | and 1000T > G | |
| (s) | 1063G > C | R |
| (t) | 1079G > A | R |
| (u) | 1085delC, 1086delA, 1087delG, | R |
| | 1089G > A, 1090T > C, 1091T > C | |
| | and 1092G > T | |
| (v) | 1161C > A, 1162T > C, 1163delA | R |
| | and 1164delT | |
| (w) | 1215C > T, 1216delG, 1217delG, | R |
| | 1218delG, 1219delG, 1220delT, | |
| | 1221delG, 1222delG, 1223delA, | |
| | 1224delG, 1225delG, 1226delG, | |
| | 1227delG, 1228delA, 1229delG, | |
| | 1230delA, 1231delT, 1232delA, | |
| | 1233delG, 1234delA, 1235delC, | |
| | 1236delC and 1237C > T | |
| (x) | Wild type | S |

Sensitive (S); Resistant (R).

### 2.A. General Methods

Once a subject is identified as being at risk for developing or having a cancer or a SPARC-inhibited cancer, genetic sequence information may be obtained from the subject. Various methods for obtaining biological samples from a subject that contain genetic material, are known in the art. For example, tissue samples may be obtained by curettage, needle aspiration biopsy or needle (core) biopsy, incisional biopsy for sampling a tumor, or excisional biopsy, which may entail total removal of the tissue of interest. Alternatively, lo other bodily samples that contain genetic material, such as hair, sputum, urine, stool, semen or blood may be collected using methods known in the art.

Genetic sequence information may be obtained from a biological sample containing genetic material in numerous different ways, particularly, genetic material containing the sequence or sequences of interest. Many methods are known in the art for extracting genetic material from biological samples. There are many known methods for the separate isolation of DNA and RNA from biological samples. Typically, DNA may be isolated from a biological sample when first the sample is lysed and then the DNA is isolated from the lysate according to any one of a variety of multi-step protocols, which can take varying lengths of time. DNA isolation methods may involve the use of phenol (SAMBROOK, J. et al. “Molecular Cloning”, Vol. 2, pp. 9.14-9.23, Cold Spring Harbor Laboratory Press (1989) and AUSUBEL, F M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in VAN NESS et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in KOLLER (U.S. Pat. No. 5,128,247).

Yet another method of DNA isolation, which is described by BOTWELL, D D L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in the SPARC promoter sequence, as described in TABLE 1. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Mutation detection methods may include but are not limited to the following: Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

Sequencing—For example the Maxam-Gilbert technique for sequencing (MAXAM A M. and GILBERT W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al. (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Qβ replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4): 1760-1764). Other methods include those of Donis-Keller et al. (1977, Nucl. Acids Res. 4:2527-2538), SIMONCSITS A. et al. (Nature (1977) 269(5631):833-836), AXELROD V D. et al. (Nucl. Acids Res.(1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-termiinator incorporation with fluorescent polarization-detection (TDI-FP) method is described by FREEMAN B D. et al. (J Mol Diagnostics (2002) 4(4):209-215) for large scale screening;

Oligonucleotide ligation assay (OLA) is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2):131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8.);

Polymerase proofreading methods are used to determine SNPs identities, as described in WO 0181631;

Detection of single base pair DNA mutations by enzyme-amplified electronic transduction is described in PATOLSKY F et al. Nat Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70); Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158; LITTLE D P. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Provided herein are methods of detecting mutations, which result in an altered levels of SPARC polypeptide. “Altered levels” as used herein include, e.g., a change of about 10%, a change of about 20%, a change of about 30%, a change of about 40%, a change of about 50%, a change of about 75%, and a change of about 100% as compared to wild type levels for the same cell or tissue type. “Altered levels” as used herein also include, e.g., an at least about 2 fold, an at least about 5 fold, an at least about 10 fold, an at least about 20 fold, an at least about 50 fold, an at least about 100 fold, an at least about 200 fold, an at least about 500 fold, and an at least about 1,000 fold change in the level as compared to wild type levels for the same cell or tissue type. Accordingly, included is a method of detecting a mutation in a SPARC sequence occurring in an animal, wherein the mutation is in the promoter of the gene and results in an altered intracellular and/or extracellular levels of a polypeptide encoded by the SPARC gene. The method in accordance with the invention further provides for the detection of mutations wherein the mutation is a point mutation, deletion, insertion or frameshift mutation or combinations thereof. In addition, mutations detectable with the methods described include, e.g., deletion and/or insertions of 1 nucleotide, from 1 to about 5 nucleotides, from about 5 to about 10 nucleotides, from about 10 to about 20 nucleotides, from about 20 to about 50 nucleotides, and from about 50 to about 100 nucleotides, from about 100 nucleotides to about 1000 nucleotides. Altered levels as used herein may be a reduction in the intracellular and/or extracellular SPARC polypeptide.

Mutation detection in accordance with the methods described herein may include the determination of SPARC promoter sequence of at least about 1 nucleotide, alternatively at least about 5 nucleotides, at least about 10 nucleotides, at least about 50 nucleotides, at least about 100 nucleotides, at least about 200 nucleotides, at least about 300 nucleotides, at least about 400 nucleotides, at least about 500 nucleotides, at least about 1,000 nucleotides, or at least about 2,000 nucleotides.

Provided herein are nucleic acid molecules with one or more SPARC gene promoter mutations, further including a reporter sequence and/or further including a vector. Suitable nucleic acid molecules may include molecules of at least about 10 base pairs, at least about 50 base pairs, at least about 100 base pairs, at least about 200 base pairs, at least about 300 base pairs, at least about 500 base pairs, and at least about 1,000 base pairs. In addition, there is provided a cell including the nucleic acid molecule with a mutant SPARC promoter genotype.

Alternatively, if a subject's genetic sequence data is already known, then obtaining may involve retrieval of the subject's nucleic acid sequence data from a database, followed by determining or detecting the identity of a nucleic acid or genotype at a polymorphism site by reading the subject's nucleic acid sequence at the polymorphic site.

Once the identity of a polymorphism(s) or other mutation is determined or detected an indication may be obtained as to resistance of a subject's cancer to a therapeutic regimen based on the based on the presence or absence of one or more mutations in the subject's SPARC promoter sequence. As described herein, polymorphisms or other mutations in the SPARC promoter sequence, may used to obtain a prognosis or to make a determination regarding the ability of the subject to have a favourable outcome following a chemotherapy, radiotherapy or combination therapy regiment for a cancer. Specifically, to determine whether the subject's cancer is likely to be sensitive to a particular therapeutic regimen or whether the cancer is likely to be resistant to a particular therapeutic regimen. Alternatively, polymorphisms or other mutations in the SPARC promoter may be used to screen subjects to determine the ability of a subject to be treated by a chemotherapy, radiotherapy or combination therapy regimen for cancer. Examples may include subjects to be included in a clinical trial or other similar study. Alternatively, mutations may be used as an indication of a subject's ability to respond to a chemotherapy, radiotherapy or combination therapy regimen directed at the subject's cancer. A mutation may be indicative of a subject's cancer being resistant to a chemotherapy, radiotherapy or combination therapy regimen. Accordingly, a decision regarding the subject's chemotherapy, radiotherapy or combination therapy regimen may be made based on the subject's SPARC gene sequence.

Once a subject's SPARC promoter sequence or SPARC mutational status is determined, such information may be of interest to physicians and surgeons to assist in deciding between potential treatment options, to help determine the degree to which subjects are monitored and the frequency with which such monitoring occurs. Ultimately, treatment decisions may be made in response to factors, both specific to the subject and based on the experience of the physician or surgeon responsible for a subject's care. For example, a subject who has one or more SPARC promoter mutations that are indicative of resistance to a particular therapeutic regimen, may be offered an “alternative therapeutic regimen”. Accordingly, a subject who is receiving a first line chemotherapeutic regimen (or is about to receive such a regimen) and has one or more SPARC promoter mutations in their cancer may be preferentially administered an alternative therapeutic regimen. For example, SPARC polypeptide (as described in WO 2004/064785), receptor tyrosine kinase inhibitors (for example Iressa™ (gefitinib), Tarceva™ (erlotinib), Erbitux™ (cetuximab), imatinib mesilate (Gleeve) or Avastin™ (bevacizumab), proteosome inhibitors (for example bortezomib, Velcade); aurora kinase inhibitors (for example ZM447439); rapamycin inhibitors (for example sirolimus, Rapamune); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); TNP-4, a synthetic analogue of fumagillin; carboxyaminotriazole; BB-94 and BB-2516; thalidomide; interleukin-12; linomide; peptide fragments; and antibodies to vascular growth factors and vascular growth factor receptors); cyclo-oxygenase inhibitors, platelet derived growth factor receptor inhibitors, protein kinase C inhibitors, mitogen-activated protein kinase kinase inhibitors, histonedeacetylase inhibitors, small hypoxia-inducible factor inhibitors, hedgehog inhibitors, and TGF-β signalling inhibitors. Furthermore, “Immunotherapeutic agent”, as used herein, would also be considered an alternative therapeutic regimen. For example, agents used to transfer the immunity of an immune donor, e.g., another person or an animal, to a host by inoculation. The term embraces the use of serum or gamma globulin containing performed antibodies produced by another individual or an animal; nonspecific systemic stimulation; adjuvants; active specific immunotherapy; and adoptive immunotherapy. Adoptive immunotherapy refers to the treatment of a disease by therapy or agents that include host inoculation of sensitized lymphocytes, transfer factor, immune RNA, or antibodies in serum or gamma globulin. Such alternative therapeutics may be administered alone or in combination or in combination with other therapeutic regimens described herein.

A subject may be monitored on an ongoing basis for SPARC promoter mutations during the course of chemotherapy, radiotherapy or combination therapy regimen. A response to a selected regimen may be predicted by the presence or absence of one or more SPARC promoter mutations as described herein. An improved response may include an increased likelihood of survival, reduction in tumor size or reduction in the rate of metastasis. Tumor staging provides a method to assess the size and spread, and by extension guide prognosis of a tumor in a subject. The TNM tumor staging system uses three components to express the anatomic extent of disease: T is a measure of the local extend of tumor spread (size), N indicates the presence or absence of metastatic spread to regional lymph nodes, and M specifies the presence or absence of metatstatic spread to distant sites. The combination of these three classifications combine to provide a stage grouping. Clinical TNM (cTNM) defines the tumor based on clinical evidence. Pathologic TNM (pTNM) defines the tumor based on examination of a surgically resected specimen.

Changes in tumor size may be observed by various imaging methods known to physicians or surgeons in the field of oncology therapy and diagnostics. Examples of imaging methods include positron emission tomography (PET) scanning, computed tomography (CT) scanning, PET/CT scanning, magnetic resonance imaging (MRI), chemical shift imaging, radiography, bone-scan, mammography, fiberoptic colonoscopy or ultrasound. Contrast agents, tracers and other specialized techniques may also be employed to image specific types of cancers, or for particular organs or tissues, and will be known to those skilled in the art. Changes in rate of metastasis may also be observed by the various imaging methods, considering particularly the appearance, or frequency of appearance, of tumors distal to the primary site. Alternatively, the presence of tumor cells in lymph nodes adjacent and distal to the primary tumor site may also be detected and used to monitor metastasis.

### 2.B. Methods and Materials

### Cell Culture and Maintenance

A human colorectal cancer cell line, MIP-101, and uterine sarcoma cell line, MES-SA were maintained in Dulbecco's modified Eagle's medium (DMEM) supplemented with 10% fetal bovine serum (FBS), 1% penicillin/streptomycin (Invitrogen, Carlsbad, Calif.) at 37° C. and 5% CO₂. MIP/5FU (MIP-101 cells resistant to 5-fluorouracil), MIP/CIS (MIP-101 cells resistant to cisplatin) and MIP/CPT (MIP-101 cells resistant to irinotecan) were maintained as described above, but with the inclusion of the respective resistant drug (5-flurouracil 200 μM, cisplatin 10 μM, irinotecan 20 μM). MES-SA cells resistant to doxorubicin (MES-SA/DX5) were acquired from ATCC (# CRL-1977) and maintained according to supplier's recommendations.

### Development of Resistant Cell Lines

Resistant MIP101 cells were developed following long-term incubation with incremental concentrations of chemotherapeutic agents 5-fluorouracil (5FU; 10-500 μM) (Sigma, St. Louis, Mo.), cisplatin (CIS; 1-100 μM) (Sigma, St. Louis, Mo.) or irinotecan (CPT; 1-50 μM) (Pharmacia, Kalamazoo, Mich.) over 3 months. The concentration of each chemotherapeutic agent was increased by 10% every 2 weeks for a 6 month period.

### Isolation of Cellular DNA

10-20 million colorectal cancer cells from sensitive (MIP 101, MES-SA) and resistant (MIP/5FU, MIP/CPT, MIP/CIS, MES-SA/DX5) cells were collected and DNA extracted based on the manufacturer's protocol (DNA Extraction Kit, Gentra Systems, Inc.).

### Sequencing Reactions

Overlapping sections of the SPARC promoter were sequenced using the BigDye™ Terminator v 3.1 Cycle Sequencing Kit (BD Biosciences) following manufacturer's instructions. Briefly, 4 μl of reaction premix, 2 μl of sequencing buffer, 3.2 pmol of primer and 50-100 ng of isolated cellular DNA was combined with water to make a final volume of 20 μl. Cycle sequencing was performed using the following parameters: an initial denaturation step at 95° C. for 2 minutes, followed by 30 cycles of 95° C. for 30 seconds, 60° C. for 30 seconds, 72° C. for 1 minute, with a final extension at 72° C. for 10 min. Sequencing reaction amplicons were purified by incubation with AMPure magnetic beads (Agencourt Bioscience Corporation), following manufacturer's instructions. Briefly, the sequencing reaction mixture was incubated with AMPure magnetic beads for 10 min, followed by collection of beads with a magnetic plate. The collected beads were washed once with 70% ethanol, and the PCR amplicons eluted with Tris-EDTA, ph 8.0. The eluted sequencing reaction amplicons were sequenced using an ABI Prism 3700 DNA analyzer. Sequence chromatograms generated by ABI Prism 3700 DNA Analyzer were analyzed and assembled by Phred-Phrap programs and assembled by Consed program using reference sequences from UCSC Bioinformatics for the identification of mutations.

### Analytical RT-PCR.

To determine SPARC mRNA levels Analytical RT-PCR could be performed as described by TAI, I T. et al. (J. Clin Invest. (2005) 115:1493-1502). Briefly, total RNA would be extracted from cultured cells, at 75% confluence and under similar culture conditions, using TRIZOL reagent, following manufacturer's instructions. RT-PCR could be performed using One-Step RT-PCR (BD Biosciences), following manufacturer's instructions. SPARC-specific primers such as SEQ ID NO: 52 and SEQ ID NO: 53. GAPDH-specific primers SEQ ID NO: 54 and SEQ ID NO: 55 could be used. The reaction conditions for such a reaction would be 50° C. for 1 hour, followed by 45 cycles at 94° C. for 1 minute, 65° C. for 1 minute, 72° C. for 2 minutes, and 72° C. for 10 minutes. PCR products could then be separated on 1% agarose gel electrophoresis.

### Protein Extracts and Immunoblots

To determine SPARC protein levels, extracts and immunoblots could be prepared as described by TAI I T et al. 2005, supra. Briefly, total protein could be extracted from cell lysates using CHAPS lysis buffer (50 mM PIPES/HCl, pH 6.5; 2 mM EDTA; 0.1% CHAPS; 20 μg/ml Leupeptin; 10 μg/ml Pepstatin A; 10 μg/ml Aprotinin; 5 mM DTT; 1 mM PMSF). The proteins could be resolved by SDS-PAGE (10% gel) under reducing conditions and electrotransferred onto a PVDF membrane and analyzed by Western blot. Antibodies to SPARC (1 μg/ml; Haematologic Technologies Inc.) and alpha-tubulin (0.2 μg/ml; Sigma-Aldrich) could be used.

SPARC mRNA and protein levels have been reported to be low in cell lines resistant to chemotherapy. TAI I T. et al. 2005, supra describe decreased SPARC mRNA and protein levels in MIP resistant cells (MIP/5FU, MIP/CPT, MIP/CIS and MES-SA/DX5, among others), as evaluated by RT-PCR and western blotting. Significant reductions in SPARC-specific mRNA compared with the sensitive parental lines was observed for all cell lines tested. This result was of particular interest as it was consistent over both colorectal carcinoma and uterine sarcoma cells, resistant to four different chemotherapeutic agents. Protein levels of SPARC were also significantly decreased in the resistant cell lines, again reflected in both colorectal carcinoma and uterine sarcoma cell lines, resistant to four different chemotherapeutic agents.

### 2. EXAMPLES

### Example 1

### Mutations in SPARC Regulatory Regions in MIP/5FU Cell Lines

A region spanning 2963 base pairs spanning a regulatory region of SPARC ˜1400 bp upstream of exon 1, exon 1 and ˜1300 of intron 1 (SEQ ID NO: 9) was sequenced on both strands using primers corresponding to SEQ ID NOs: 34 to 39 in both sensitive (MIP-101) and 5FU resistant (MIP/5FU) cell lines. Several mutations were identified in the regulatory region between nucleotides 1 to 1370 of SEQ ID NO: 1 that were not found in the sensitive cell line. The individual mutations are listed in TABLE 1—mutations specifically identified to date in the MIP/5FU cell lines are shown in TABLE 5 infra.

### Example 2

### Mutations in SPARC Regulatory Regions in MIP/CPT Cell Lines

A region spanning 2963 base pairs spanning a regulatory region of SPARC ˜1400 bp upstream of exon 1, exon 1 and ˜1300 of intron 1 (SEQ ID NO: 9) was sequenced on both strands using primers corresponding to SEQ ID NOs: 34-39 in both sensitive (MIP-101) and 5FU resistant (MIP/5FU) cell lines. Several mutations were identified in the regulatory region between nucleotides 1 to 1370 of SEQ ID NO: 1 that were not found in the sensitive cell line. The individual mutations are listed in TABLE 1—mutations specifically identified to date in the MIP/CPT cell lines are shown in TABLE 5 infra.

### Example 3

### Mutations in SPARC Regulatory Regions in MIP/CIS Cell Lines

A region spanning 2963 base pairs spanning a regulatory region of SPARC ˜1400 bp upstream of exon 1, exon 1 and ˜1300 of intron 1 (SEQ ID NO: 9) was sequenced on both strands using primers corresponding to SEQ ID NOs: 34-39 in both sensitive (MIP-101) and 5FU resistant (MIP/5FU) cell lines. Several mutations were identified in the regulatory region between nucleotides 1 to 1370 of SEQ ID NO: 1 that were not found in the sensitive cell line. The individual mutations are listed in TABLE 1—mutations specifically identified to date in the MIP/CIS cell lines are shown in TABLE 5 infra.

### Example 4

### Mutations in SPARC Regulatory Regions in MES-SA/DX5 Cell Lines

A region spanning 2963 base pairs spanning a regulatory region of SPARC ˜1400 bp upstream of exon 1, exon 1 and ˜1300 of intron 1 (SEQ ID NO: 9) was sequenced on both strands using primers corresponding to SEQ ID NOs: 34-39 in both sensitive (MIP-101) and 5Fu resistant (MIP/5FU) cell lines. Several mutations were identified in the regulatory region between nucleotides 1 to 1370 of SEQ ID NO: 1 that were not found in the sensitive cell line. The individual mutations are listed in TABLE 1—mutations specifically identified to date in the MES-SAIDX5 cell lines are shown in TABLE 5 infra. The results shown in TABLE 5 below represent mutations identified to date in various drug resistant cell lines and the absence of a mutation (i.e. no √) is not meant to represent that such a mutation is not possible or even likely, only that to date not such mutation has been identified in a given resistant cell line with a given chemotherapeutic regimen.

TABLE 5

| | | Resistant Cell Lines | | | |
|---|---|---|---|---|---|
| | | (MIP = colorectal cancer, MES - uterine sarcoma) | | | |
| | | Cell Line/Therapeutic Regimen | | | |
| Mutation | SPARC MUTATIONS | MIP/ | MIP/ | MIP/ | MES/ |
| Identifier | RELATIVE TO SEQ ID NO: 1 | 5FU | CPT | cisplatin | doxorubicin |
| (a) | 295delG and 301A > CC | | | ✓ | |
| (b) | 315insA and 316G > A | | | ✓ | |
| (c) | 341C > T, 342T > C, 344A > C, | | | ✓ | |
| | 347C > A, 354G > A, 356G > A, | | | | |
| | 359T > G and 363G > A | | | | |
| (d) | 395C > T and 396A > T | | ✓ | ✓ | ✓ |
| (e) | 488T > C | | ✓ | | |
| (f) | 533A > G | | | | ✓ |
| (g) | 734G > A | ✓ | ✓ | ✓ | ✓ |
| (h) | 734delG | ✓ | ✓ | ✓ | ✓ |
| (i) | 769T > A, 771G > C and 774G > C | | ✓ | | |
| (j) | 784T > C, 786T > G and 788T > A | | ✓ | | |
| (k) | 793T > G, 796T > A and 800T > C | | ✓ | | |
| (l) | 820C > A and 824T > A | | ✓ | | |
| (m) | 864T > A and 867T > A | | ✓ | | |
| (n) | 895T > G, 899T > A, 901G > A and | | ✓ | ✓ | ✓ |
| | 904G > A | | | | |
| (o) | 901G > A | ✓ | ✓ | ✓ | ✓ |
| (p) | 901G > A, 904G > A, 929A > G and | ✓ | ✓ | ✓ | |
| | 934delC | | | | |
| (q) | 922T > G, 928T > G and 935T > G | | ✓ | | |
| (r) | 982T > C, 984T > G, 985G > A, | | ✓ | | |
| | 991G > A, 995G > A, 999C > T and | | | | |
| | 1000T > G | | | | |
| (s) | 1063G > C | ✓ | ✓ | ✓ | |
| (t) | 1079G > A | ✓ | ✓ | ✓ | |
| (u) | 1085delC, 1086delA, 1087delG, | ✓ | ✓ | ✓ | |
| | 1089G > A, 1090T > C, 1091T > C | | | | |
| | and 1092G > T | | | | |
| (v) | 1161C > A, 1162T > C, 1163delA | ✓ | ✓ | ✓ | |
| | and 1164delT | | | | |
| (w) | 1215C > T, 1216delG, 1217delG, | ✓ | ✓ | ✓ | |
| | 1218delG, 1219delG, 1220delT, | | | | |
| | 1221delG, 1222delG, 1223delA, | | | | |
| | 1224delG, 1225delG, 1226delG, | | | | |
| | 1227delG, 1228delA, 1229delG, | | | | |
| | 1230delA, 1231delT, 1232delA, | | | | |
| | 1233delG, 1234delA, 1235delC, | | | | |
| | 1236delC and 1237C > T | | | | |

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for predicting responsiveness of a subject to a therapeutic regimen, the subject having, or suspected of having a cancer, the method comprising determining a presence or an absence of one or more a secreted protein, acidic, cysteine-rich (SPARC) promoter mutations in the subject's cancer, wherein said presence or absence of one or more SPARC promoter mutations is indicative of a sensitivity of the subject's cancer to the therapeutic regimen.

2. The method of claim 1, wherein the one or more SPARC promoter mutations, are selected from the following mutations relative to SEQ ID NO: 1:
(a) 295delG and 301A>CC;
(b) 315insA and 316G>A;
(c) 341C>T, 342T>C, 344A>C, 347C>A, 354G>A, 356G>A, 359T>G and 363G>A;
(d) 395C>T and 396A>T;
(e) 488T>C;
(f) 533A>G;
(g) 734G>A;
(h) 734delG;
(i) 769T>A, 771G>C and 774G>C;
(j) 784T>C, 786T>G and 788T>A;
(k) 793T>G, 796T>A and 800T>C;
(l) 820C>A and 824T>A;
(m) 864T>A and 867T>A;
(n) 895T>G, 899T>A, 901G>A and 904G>A;
(o) 901G>A;
(p) 901G>A, 904G>A, 929A>G and 934delC;
(q) 922T>G, 928T>G and 935T>G;
(r) 982T>C, 984T>G, 985G>A, 991G>A, 995G>A, 999C>T and 1000T>G;
(s) 1063G>C;
(t) 1079G>A;
(u) 1085delC, 1086delA, 1087delG, 1089G>A, 1090T>C, 1091T>C and 1092G>T;
(v) 1161C>A, 1162T>C, 1163delA and 1164delT; and
(w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T.

3. The method of claim 1, wherein the promoter mutation results in altered intracellular and/or extracellular levels of a polypeptide encoded by the SPARC promoter sequence.

4. The method of claim 1, wherein the therapeutic regimen is selected from: radiotherapy, chemotherapy or a combination thereof.

5. The method of claim 4, wherein the therapeutic regimen comprises administration of one or more chemotherapeutic agents.

6. The method of claim 5, wherein the chemotherapeutic agent is selected from 5-flurouracil, irinotecan, cisplatin, doxorubicin or combinations thereof.

7. The method of claim 2, wherein the one or more SPARC promoter mutations are indicative of the cancer's resistance to the therapeutic regimen.

8. The method of claim 1, further comprising obtaining SPARC promoter information for the subject.

9. The method of claim 1, wherein the one or more SPARC promoter mutations are determined using a nucleic acid sample from the subject.

10. The method of claim 9, further comprising obtaining the nucleic acid sample from the subject.

11. The method of claim 10, wherein the obtaining the nucleic acid sample is from a biological sample taken from the subject's cancer.

12. The method of claim 1, wherein said determining the presence or an absence of one or more SPARC promoter mutations in the subject's cancer is done using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m)ligase chain reaction assay;
(n) enzyme-amplified electronic transduction;
(o) single base pair extension assay; and
(p) reading sequence data.

13. The method of claim 1, wherein the cancer is selected from the following: carcinoma; melanoma; sarcoma; lymphoma; and leukaemia.

14. The method of claim 13, wherein the carcinoma is selected from the following: squamous cell; adeno; small cell; large cell and combinations thereof.

15. The method of claim 14, wherein the carcinoma is derived from a primary tumor located in nasopharygeal tissue, head and neck, lung, esophagus, stomach, intestine, rectum, kidney, urinary tract, prostate, testes, ovary, uterus, cervix, vagina, skin, and endocrine glands.

16. The method of claim 13, wherein the sarcoma is selected from the following: muscle; adipose; fibrous; vascular; and neural.

17. The method of claim 13, wherein the lymphoma is selected from the following: Hodgkin's Disease; and Non-Hodgkins Lymphoma.

18. The method of claim 13, wherein the leukaemia is selected from the following: lymphoid; and myeloid.

19. The method of claim 13, wherein the cancer is a colorectal carcinoma, prostate carcinoma, ovarian carcinoma or osteosarcoma.

20. The method of claim 1, wherein the cancer is a SPARC inhibited cancer.

21. The method of claim 20, wherein the SPARC inhibited cancer is selected from colorectal carcinoma, prostate carcinoma, ovarian carcinoma, osteosarcoma and uterine sarcoma.

22. A kit for detecting a mutation within a SPARC promoter in a subject's cancer to provide an indication of the subject's sensitivity to a therapeutic regimen, the kit comprising:
(a) a restriction enzyme capable of distinguishing alternate nucleotides at the mutation site; or
(b) a labeled oligonucleotide having sufficient complementary to the mutation site so as to be capable of selectively hybridizing to either the mutated or non-mutated site.

23. The kit of claim 22, wherein the mutation is selected from one or more of the following mutations relative to SEQ ID NO: 1:
(a) 295delG and 301A>CC;
(b) 315insA and 316G>A;
(c) 341C>T, 342T>C, 344A>C, 347C>A, 354G>A, 356G>A, 359T>G and 363G>A;
(d) 395C>T and 396A>T;
(e) 488T>C;
(f) 533A>G;
(g) 734G>A;
(h) 734delG;
(i) 769T>A, 771G>C and 774G>C;
(j) 784T>C, 786T>G and 788T>A;
(k) 793T>G, 796T>A and 800T>C;
(l) 820C>A and 824T>A;
(m) 864T>A and 867T>A;
(n) 895T>G, 899T>A, 901G>A and 904G>A;
(o) 901G>A;
(p) 901G>A, 904G>A, 929A>G and 934delC;
(q) 922T>G, 928T>G and 935T>G;
(r) 982T>C, 984T>G, 985G>A, 991G>A, 995G>A, 999C>T and 1000T>G;
(s) 1063G>C;
(t) 1079G>A;
(u) 1085delC, 1086delA, 1087delG, 1089G>A, 1090T>C, 1091T>C and 1092G>T;
(v) 1161C>A, 1162T>C, 1163delA and 1164delT; and
(w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T.

24. The kit of claim 22 further comprising an oligonucleotide or a set of oligonucleotides operable to amplify a region including the mutation.

25. The kit of claim 24, further comprising a polymerization agent.

26. The kit of claim 1, further comprising instructions for using the kit to identify mutations in a SPARC promoter sequence.

27. A method for selecting a group of subjects for determining the efficacy of a therapeutic regimen known or suspected of being useful for the treatment of cancer, the method comprising determining a genotype at one or more mutation sites in a SPARC promoter for each subject, wherein said genotype is indicative of the subject's sensitivity to a therapeutic regimen.

28. The method of claim 27 further comprising, administering the therapeutic regimen to the subjects or a subset of subjects and determining each subject's sensitivity to the therapeutic regimen.

29. The method of claim 28, further comprising comparing subject response to the therapeutic regimen based on genotype of the subject.

30. The method of claim 27, wherein the therapeutic regimen is a chemotherapy.

31. The method or claim 30, wherein the chemotherapy comprises administration of 5-flurouracil, irinotecan, cisplatin, doxorubicin or combinations thereof.

32. An oligonucleotide or peptide nucleic acid of about 10 to about 400 nucleotides that hybridizes specifically to a sequence contained in a human target sequence consisting of a subject's SPARC promoter sequence, a complementary sequence of the target sequence or RNA equivalent of the target sequence and wherein the oligonucleotide or peptide nucleic acid is operable in determining the presence or absence of a SPARC promoter mutation selected from one or more of the following mutations described relative to SEQ ID NO:1:
(a) 295delG and 301A>CC;
(b) 315insA and 316G>A;
(c) 341C>T, 342T>C, 344A>C, 347C>A, 354G>A, 356G>A, 359T>G and 363G>A;
(d) 395C>T and 396A>T;
(e) 488T>C;
(f) 533A>G;
(g) 734G>A;
(h) 734delG;
(i) 769T>A, 771G>C and 774G>C;
(j) 784T>C, 786T>G and 788T>A;
(k) 793T>G, 796T>A and 800T>C;
(l) 820C>A and 824T>A;
(m) 864T>A and 867T>A;
(n) 895T>G, 899T>A, 901G>A and 904G>A;
(o) 901G>A;
(p) 901G>A, 904G>A, 929A>G and 934delC;
(q) 922T>G, 928T>G and 935T>G;
(r) 982T>C, 984T>G, 985G>A, 991G>A, 995G>A, 999C>T and 1000T>G;
(s) 1063G>C;
(t) 1079G>A;
(u) 1085delC, 1086delA, 1087delG, 1089G>A, 1090T>C, 1091T>C and 1092G>T;
(v) 1161C>A, 1162T>C, 1163delA and 1164delT; and
(w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T.

33. A nucleic acid comprising a nucleic acid corresponding to SEQ. ID NO: 1 with one or more of the following mutations:
(a) 295delG and 301A>CC;
(b) 315insA and 316G>A;
(c) 341C>T, 342T>C, 344A>C, 347C>A, 354G>A, 356G>A, 359T>G and 363G>A;
(d) 395C>T and 396A>T;
(e) 488T>C;
(f) 533A>G;
(g) 734G>A;
(h) 734delG;
(i) 769T>A, 771G>C and 774G>C;
(j) 784T>C, 786T>G and 788T>A;
(k) 793T>G, 796T>A and 800T>C;
(l) 820C>A and 824T>A;
(m) 864T>A and 867T>A;
(n) 895T>G, 899T>A, 901G>A and 904G>A;
(o) 901G>A;
(p) 901G>A, 904G>A, 929A>G and 934delC;
(q) 922T>G, 928T>G and 935T>G;
(r) 982T>C, 984T>G, 985G>A, 991G>A, 995G>A, 999C>T and 1000T>G;
(s) 1063G>C;
(t) 1079G>A;
(u) 1085delC, 1086delA, 1087delG, 1089G>A, 1090T>C, 1091T>C and 1092G>T;
(v) 1161C>A, 1162T>C, 1163delA and 1164delT; and
(w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T.

34. The nucleic acid sequence of claim 33, further comprising a reporter sequence.

35. The nucleic acid sequence of claim 33, further comprising a vector.

36. A cell comprising the nucleic acid of claim 33.

37. An oligonucleotide or peptide nucleic acid selected from the group consisting of:
(a) an oligonucleotide or peptide nucleic acid that hybridizes under high stringency conditions to a nucleic acid molecule comprising SEQ ID NO:2 having a G at position 295 but not to a nucleic acid molecule comprising SEQ ID NO:2 having a A at position 295;
(b) an oligonucleotide or peptide nucleic acid that hybridizes under high stringency conditions to a nucleic acid molecule comprising SEQ ID NO:2 having a A at position 295 but not to a nucleic acid molecule comprising SEQ ID NO:2 having a G at position 295;
(c) an oligonucleotide or peptide nucleic acid that hybridizes under high stringency conditions to a nucleic acid molecule comprising SEQ ID NO:2 having a A at position 298 but not to a nucleic acid molecule comprising SEQ ID NO:2 having a C at position 298;
(d) an oligonucleotide or peptide nucleic acid that hybridizes under high stringency conditions to a nucleic acid molecule comprising SEQ ID NO:2 having a C at position 298 but not to a nucleic acid molecule comprising SEQ ID NO:2 having a A at position 298;
(e) an oligonucleotide or peptide nucleic acid that hybridizes under high stringency conditions to a nucleic acid molecule comprising SEQ ID NO:2 having a A at position 301 but not to a nucleic acid molecule comprising SEQ ID NO:2 having a C at position 301;
(f) an oligonucleotide or peptide nucleic acid that hybridizes under high stringency conditions to a nucleic acid molecule comprising SEQ ID NO:2 having a C at position 301 but not to a nucleic acid molecule comprising SEQ ID NO:2 having a A at position 301;
(g) an oligonucleotide or peptide nucleic acid that hybridizes under high stringency conditions to a nucleic acid molecule comprising SEQ ID NO:2 having a C at position 341 but not to a nucleic acid molecule comprising SEQ ID NO:2 having a T at position 341;
(h) an oligonucleotide or peptide nucleic acid that hybridizes under high stringency conditions to a nucleic acid molecule comprising SEQ ID NO:2 having a T at position 341 but not to a nucleic acid molecule comprising SEQ ID NO:2 having a C at position 341;
(i) an oligonucleotide or peptide nucleic acid that hybridizes under high stringency conditions to a nucleic acid molecule comprising SEQ ID NO:2 having a T at position 342 but not to a nucleic acid molecule comprising SEQ ID NO:2 having a C at position 342;
(j) an oligonucleotide or peptide nucleic acid that hybridizes under high stringency conditions to a nucleic acid molecule comprising SEQ ID NO:2 having a C at position 342 but not to a nucleic acid molecule comprising SEQ ID NO:2 having a T at position 342;
(k) an oligonucleotide or peptide nucleic acid that hybridizes under high stringency conditions to a nucleic acid molecule comprising SEQ ID NO:2 having a A at position 344 but not to a nucleic acid molecule comprising SEQ ID NO:2 having a C at position 344;
(l) an oligonucleotide or peptide nucleic acid that hybridizes under high stringency conditions to a nucleic acid molecule comprising SEQ ID NO:2 having a C at position 344 but not to a nucleic acid molecule comprising SEQ ID NO:2 having a A at position 344;
(m) an oligonucleotide or peptide nucleic acid that hybridizes under high stringency conditions to a nucleic acid molecule comprising SEQ ID NO:2 having a C at position 347 but not to a nucleic acid molecule comprising SEQ ID NO:2 having a A at position 347;
(n) an oligonucleotide or peptide nucleic acid that hybridizes under high stringency conditions to a nucleic acid molecule comprising SEQ ID NO:2 having a A at position 347 but not to a nucleic acid molecule comprising SEQ ID NO:2 having a C at position 347;
(o) an oligonucleotide or peptide nucleic acid that hybridizes under high stringency conditions to a nucleic acid molecule comprising SEQ ID NO:2 having a G at position 354 but not to a nucleic acid molecule comprising SEQ ID NO:2 having a A at position 354;
(p) an oligonucleotide or peptide nucleic acid that hybridizes under high stringency conditions to a nucleic acid molecule comprising SEQ ID NO:2 having a A at position 354 but not to a nucleic acid molecule comprising SEQ ID NO:2 having a G at position 354;
(q) an oligonucleotide or peptide nucleic acid that hybridizes under high stringency conditions to a nucleic acid molecule comprising SEQ ID NO:2 having a G at position 356 but not to a nucleic acid molecule comprising SEQ ID NO:2 having a A at position 356;
(r) an oligonucleotide or peptide nucleic acid that hybridizes under high stringency conditions to a nucleic acid molecule comprising SEQ ID NO:2 having a A at position 356 but not to a nucleic acid molecule comprising SEQ ID NO:2 having a G at position 356;
(s) an oligonucleotide or peptide nucleic acid that hybridizes under high stringency conditions to a nucleic acid molecule comprising SEQ ID NO:2 having a T at position 359 but not to a nucleic acid molecule comprising SEQ ID NO:2 having a G at position 359;
(t) an oligonucleotide or peptide nucleic acid that hybridizes under high stringency conditions to a nucleic acid molecule comprising SEQ ID NO:2 having a G at position 359 but not to a nucleic acid molecule comprising SEQ ID NO:2 having a T at position 359;
(u) an oligonucleotide or peptide nucleic acid that hybridizes under high stringency conditions to a nucleic acid molecule comprising SEQ ID NO:2 having a G at position 363 but not to a nucleic acid molecule comprising SEQ ID NO:2 having a A at position 363;
(v) an oligonucleotide or peptide nucleic acid that hybridizes under high stringency conditions to a nucleic acid molecule comprising SEQ ID NO:2 having a A at position 363 but not to a nucleic acid molecule comprising SEQ ID NO:2 having a G at position 363;
(w) an oligonucleotide or peptide nucleic acid that hybridizes under high stringency conditions to a nucleic acid molecule comprising SEQ ID NO:2 having a C at position 395 but not to a nucleic acid molecule comprising SEQ ID NO:2 having a T at position 395;
(x) an oligonucleotide or peptide nucleic acid that hybridizes under high stringency conditions to a nucleic acid molecule comprising SEQ ID NO:2 having a T at position 395 but not to a nucleic acid molecule comprising SEQ ID NO:2 having a C at position 395;
(y) an oligonucleotide or peptide nucleic acid that hybridizes under high stringency conditions to a nucleic acid molecule comprising SEQ ID NO:2 having a A at position 396 but not to a nucleic acid molecule comprising SEQ ID NO:2 having a T at position 396;
(z) an oligonucleotide or peptide nucleic acid that hybridizes under high stringency conditions to a nucleic acid molecule comprising SEQ ID NO:2 having a T at position 396 but not to a nucleic acid molecule comprising SEQ ID NO:2 having a A at position 396;
(aa) an oligonucleotide or peptide nucleic acid that hybridizes under high stringency conditions to a nucleic acid molecule comprising SEQ ID NO:2 having a T at position 488 but not to a nucleic acid molecule comprising SEQ ID NO:2 having a C at position 488;
(bb) an oligonucleotide or peptide nucleic acid that hybridizes under high stringency conditions to a nucleic acid molecule comprising SEQ ID NO:2 having a C at position 488 but not to a nucleic acid molecule comprising SEQ ID NO:2 having a T at position 488;
(cc) an oligonucleotide or peptide nucleic acid that hybridizes under high stringency conditions to a nucleic acid molecule comprising SEQ ID NO:2 having a A at position 533 but not to a nucleic acid molecule comprising SEQ ID NO:2 having a G at position 533;
(dd) an oligonucleotide or peptide nucleic acid that hybridizes under high stringency conditions to a nucleic acid molecule comprising SEQ ID NO:2 having a G at position 533 but not to a nucleic acid molecule comprising SEQ ID NO:2 having a A at position 533;
(ee) an oligonucleotide or peptide nucleic acid that hybridizes under high stringency conditions to a nucleic acid molecule comprising SEQ ID NO:2 having a G at position 734 but not to a nucleic acid molecule comprising SEQ ID NO:2 having a A at position 734;
(ff) an oligonucleotide or peptide nucleic acid that hybridizes under high stringency conditions to a nucleic acid molecule comprising SEQ ID NO:2 having a A at position 734 but not to a nucleic acid molecule comprising SEQ ID NO:2 having a G at position 734;
(gg) an oligonucleotide or peptide nucleic acid that hybridizes under high stringency conditions to a nucleic acid molecule comprising SEQ ID NO:2 having a T at position 769 but not to a nucleic acid molecule comprising SEQ ID NO:2 having a A at position 769;
(hh) an oligonucleotide or peptide nucleic acid that hybridizes under high stringency conditions to a nucleic acid molecule comprising SEQ ID NO:2 having a A at position 769 but not to a nucleic acid molecule comprising SEQ ID NO:2 having a T at position 769;
(ii) an oligonucleotide or peptide nucleic acid that hybridizes under high stringency conditions to a nucleic acid molecule comprising SEQ ID NO:2 having a G at position 771 but not to a nucleic acid molecule comprising SEQ ID NO:2 having a C at position 771;
(jj) an oligonucleotide or peptide nucleic acid that hybridizes under high stringency conditions to a nucleic acid molecule comprising SEQ ID NO:2 having a C at position 771 but not to a nucleic acid molecule comprising SEQ ID NO:2 having a G at position 771;
(kk) an oligonucleotide or peptide nucleic acid that hybridizes under high stringency conditions to a nucleic acid molecule comprising SEQ ID NO:2 having a G at position 774 but not to a nucleic acid molecule comprising SEQ ID NO:2 having a C at position 774;
(ll) an oligonucleotide or peptide nucleic acid that hybridizes under high stringency conditions to a nucleic acid molecule comprising SEQ ID NO:2 having a C at position 774 but not to a nucleic acid molecule comprising SEQ ID NO:2 having a G at position 774;
(mm) an oligonucleotide or peptide nucleic acid that hybridizes under high stringency conditions to a nucleic acid molecule comprising SEQ ID NO:2 having a T at position 784 but not to a nucleic acid molecule comprising SEQ ID NO:2 having a C at position 784;
(nn) an oligonucleotide or peptide nucleic acid that hybridizes under high stringency conditions to a nucleic acid molecule comprising SEQ ID NO:2 having a C at position 784 but not to a nucleic acid molecule comprising SEQ ID NO:2 having a T at position 784;
(oo) an oligonucleotide or peptide nucleic acid that hybridizes under high stringency conditions to a nucleic acid molecule comprising SEQ ID NO:2 having a T at position 786 but not to a nucleic acid molecule comprising SEQ ID NO:2 having a G at position 786;
(pp) an oligonucleotide or peptide nucleic acid that hybridizes under high stringency conditions to a nucleic acid molecule comprising SEQ ID NO:2 having a G at position 786 but not to a nucleic acid molecule comprising SEQ ID NO:2 having a T at position 786;
(qq) an oligonucleotide or peptide nucleic acid that hybridizes under high stringency conditions to a nucleic acid molecule comprising SEQ ID NO:2 having a T at position 788 but not to a nucleic acid molecule comprising SEQ ID NO:2 having a A at position 788;
(rr) an oligonucleotide or peptide nucleic acid that hybridizes under high stringency conditions to a nucleic acid molecule comprising SEQ ID NO:2 having a A at position 788 but not to a nucleic acid molecule comprising SEQ ID NO:2 having a T at position 788;
(ss) an oligonucleotide or peptide nucleic acid that hybridizes under high stringency conditions to a nucleic acid molecule comprising SEQ ID NO:2 having a T at position 796 but not to a nucleic acid molecule comprising SEQ ID NO:2 having a G at position 796;
(tt) an oligonucleotide or peptide nucleic acid that hybridizes under high stringency conditions to a nucleic acid molecule comprising SEQ ID NO:2 having a G at position 796 but not to a nucleic acid molecule comprising SEQ ID NO:2 having a T at position 796;
(uu) an oligonucleotide or peptide nucleic acid that hybridizes under high stringency conditions to a nucleic acid molecule comprising SEQ ID NO:2 having a T at position 800 but not to a nucleic acid molecule comprising SEQ ID NO:2 having a C at position 800;
(vv) an oligonucleotide or peptide nucleic acid that hybridizes under high stringency conditions to a nucleic acid molecule comprising SEQ ID NO:2 having a C at position 800 but not to a nucleic acid molecule comprising SEQ ID NO:2 having a T at position 800;
(ww) an oligonucleotide or peptide nucleic acid that hybridizes under high stringency conditions to a nucleic acid molecule comprising SEQ ID NO:2 having a C at position 820 but not to a nucleic acid molecule comprising SEQ ID NO:2 having a A at position 820;
(xx) an oligonucleotide or peptide nucleic acid that hybridizes under high stringency conditions to a nucleic acid molecule comprising SEQ ID NO:2 having a A at position 820 but not to a nucleic acid molecule comprising SEQ ID NO:2 having a C at position 820;
(yy) an oligonucleotide or peptide nucleic acid that hybridizes under high stringency conditions to a nucleic acid molecule comprising SEQ ID NO:2 having a T at position 824 but not to a nucleic acid molecule comprising SEQ ID NO:2 having a A at position 824;
(zz) an oligonucleotide or peptide nucleic acid that hybridizes under high stringency conditions to a nucleic acid molecule comprising SEQ ID NO:2 having a A at position 824 but not to a nucleic acid molecule comprising SEQ ID NO:2 having a T at position 824;
(aaa) an oligonucleotide or peptide nucleic acid that hybridizes under high stringency conditions to a nucleic acid molecule comprising SEQ ID NO:2 having a T at position 864 but not to a nucleic acid molecule comprising SEQ ID NO:2 having a A at position 864;
(bbb) an oligonucleotide or peptide nucleic acid that hybridizes under high stringency conditions to a nucleic acid molecule comprising SEQ ID NO:2 having a A at position 864 but not to a nucleic acid molecule comprising SEQ ID NO:2 having a T at position 864;
(ccc) an oligonucleotide or peptide nucleic acid that hybridizes under high stringency conditions to a nucleic acid molecule comprising SEQ ID NO:2 having a T at position 867 but not to a nucleic acid molecule comprising SEQ ID NO:2 having a A at position 867;
(ddd) an oligonucleotide or peptide nucleic acid that hybridizes under high stringency conditions to a nucleic acid molecule comprising SEQ ID NO:2 having a A at position 867 but not to a nucleic acid molecule comprising SEQ ID NO:2 having a T at position 867;
(eee) an oligonucleotide or peptide nucleic acid that hybridizes under high stringency conditions to a nucleic acid molecule comprising SEQ ID NO:2 having a T at position 895 but not to a nucleic acid molecule comprising SEQ ID NO:2 having a G at position 895;
(fff) an oligonucleotide or peptide nucleic acid that hybridizes under high stringency conditions to a nucleic acid molecule comprising SEQ ID NO:2 having a G at position 895 but not to a nucleic acid molecule comprising SEQ ID NO:2 having a T at position 895;
(ggg) an oligonucleotide or peptide nucleic acid that hybridizes under high stringency conditions to a nucleic acid molecule comprising SEQ ID NO:2 having a T at position 899 but not to a nucleic acid molecule comprising SEQ ID NO:2 having a A at position 899;
(hhh) an oligonucleotide or peptide nucleic acid that hybridizes under high stringency conditions to a nucleic acid molecule comprising SEQ ID NO:2 having a A at position 899 but not to a nucleic acid molecule comprising SEQ ID NO:2 having a T at position 899;
(iii) an oligonucleotide or peptide nucleic acid that hybridizes under high stringency conditions to a nucleic acid molecule comprising SEQ ID NO:2 having a G at position 901 but not to a nucleic acid molecule comprising SEQ ID NO:2 having a A at position 901;
(jjj) an oligonucleotide or peptide nucleic acid that hybridizes under high stringency conditions to a nucleic acid molecule comprising SEQ ID NO:2 having a A at position 901 but not to a nucleic acid molecule comprising SEQ ID NO:2 having a G at position 901;
(kkk) an oligonucleotide or peptide nucleic acid that hybridizes under high stringency conditions to a nucleic acid molecule comprising SEQ ID NO:2 having a G at position 904 but not to a nucleic acid molecule comprising SEQ ID NO:2 having a A at position 904;
(lll) an oligonucleotide or peptide nucleic acid that hybridizes under high stringency conditions to a nucleic acid molecule comprising SEQ ID NO:2 having a A at position 904 but not to a nucleic acid molecule comprising SEQ ID NO:2 having a G at position 904;
(mmm) an oligonucleotide or peptide nucleic acid that hybridizes under high stringency conditions to a nucleic acid molecule comprising SEQ ID NO:2 having a A at position 929 but not to a nucleic acid molecule comprising SEQ ID NO:2 having a G at position 929;
(nnn) an oligonucleotide or peptide nucleic acid that hybridizes under high stringency conditions to a nucleic acid molecule comprising SEQ ID NO:2 having a G at position 929 but not to a nucleic acid molecule comprising SEQ ID NO:2 having a A at position 929;
(ooo) an oligonucleotide or peptide nucleic acid that hybridizes under high stringency conditions to a nucleic acid molecule comprising SEQ ID NO:2 having a T at position 922 but not to a nucleic acid molecule comprising SEQ ID NO:2 having a G at position 922;
(ppp) an oligonucleotide or peptide nucleic acid that hybridizes under high stringency conditions to a nucleic acid molecule comprising SEQ ID NO:2 having a G at position 922 but not to a nucleic acid molecule comprising SEQ ID NO:2 having a T at position 922;
(qqq) an oligonucleotide or peptide nucleic acid that hybridizes under high stringency conditions to a nucleic acid molecule comprising SEQ ID NO:2 having a T at position 928 but not to a nucleic acid molecule comprising SEQ ID NO:2 having a G at position 928;
(rrr) an oligonucleotide or peptide nucleic acid that hybridizes under high stringency conditions to a nucleic acid molecule comprising SEQ ID NO:2 having a G at position 928 but not to a nucleic acid molecule comprising SEQ ID NO:2 having a T at position 928;
(sss) an oligonucleotide or peptide nucleic acid that hybridizes under high stringency conditions to a nucleic acid molecule comprising SEQ ID NO:2 having a T at position 935 but not to a nucleic acid molecule comprising SEQ ID NO:2 having a G at position 935;
(ttt) an oligonucleotide or peptide nucleic acid that hybridizes under high stringency conditions to a nucleic acid molecule comprising SEQ ID NO:2 having a G at position 935 but not to a nucleic acid molecule comprising SEQ ID NO:2 having a T at position 935;
(uuu) an oligonucleotide or peptide nucleic acid that hybridizes under high stringency conditions to a nucleic acid molecule comprising SEQ ID NO:2 having a T at position 982 but not to a nucleic acid molecule comprising SEQ ID NO:2 having a C at position 982;
(vvv) an oligonucleotide or peptide nucleic acid that hybridizes under high stringency conditions to a nucleic acid molecule comprising SEQ ID NO:2 having a C at position 982 but not to a nucleic acid molecule comprising SEQ ID NO:2 having a T at position 982;
(www) an oligonucleotide or peptide nucleic acid that hybridizes under high stringency conditions to a nucleic acid molecule comprising SEQ ID NO:2 having a T at position 984 but not to a nucleic acid molecule comprising SEQ ID NO:2 having a G at position 984;
(xxx) an oligonucleotide or peptide nucleic acid that hybridizes under high stringency conditions to a nucleic acid molecule comprising SEQ ID NO:2 having a G at position 984 but not to a nucleic acid molecule comprising SEQ ID NO:2 having a T at position 984;
(yyy) an oligonucleotide or peptide nucleic acid that hybridizes under high stringency conditions to a nucleic acid molecule comprising SEQ ID NO:2 having a G at position 985 but not to a nucleic acid molecule comprising SEQ ID NO:2 having a A at position 985;
(zzz) an oligonucleotide or peptide nucleic acid that hybridizes under high stringency conditions to a nucleic acid molecule comprising SEQ ID NO:2 having a A at position 985 but not to a nucleic acid molecule comprising SEQ ID NO:2 having a G at position 985;
(aaaa) an oligonucleotide or peptide nucleic acid that hybridizes under high stringency conditions to a nucleic acid molecule comprising SEQ ID NO:2 having a G at position 991 but not to a nucleic acid molecule comprising SEQ ID NO:2 having a A at position 991;
(bbbb) an oligonucleotide or peptide nucleic acid that hybridizes under high stringency conditions to a nucleic acid molecule comprising SEQ ID NO:2 having a A at position 991 but not to a nucleic acid molecule comprising SEQ ID NO:2 having a G at position 991;
(cccc) an oligonucleotide or peptide nucleic acid that hybridizes under high stringency conditions to a nucleic acid molecule comprising SEQ ID NO:2 having a G at position 995 but not to a nucleic acid molecule comprising SEQ ID NO:2 having a A at position 995;
(dddd) an oligonucleotide or peptide nucleic acid that hybridizes under high stringency conditions to a nucleic acid molecule comprising SEQ ID NO:2 having a A at position 995 but not to a nucleic acid molecule comprising SEQ ID NO:2 having a G at position 995;
(eeee) an oligonucleotide or peptide nucleic acid that hybridizes under high stringency conditions to a nucleic acid molecule comprising SEQ ID NO:2 having a C at position 999 but not to a nucleic acid molecule comprising SEQ ID NO:2 having a T at position 999;
(ffff) an oligonucleotide or peptide nucleic acid that hybridizes under high stringency conditions to a nucleic acid molecule comprising SEQ ID NO:2 having a T at position 999 but not to a nucleic acid molecule comprising SEQ ID NO:2 having a C at position 999;
(gggg) an oligonucleotide or peptide nucleic acid that hybridizes under high stringency conditions to a nucleic acid molecule comprising SEQ ID NO:2 having a T at position 1000 but not to a nucleic acid molecule comprising SEQ ID NO:2 having a G at position 1000;
(hhhh) an oligonucleotide or peptide nucleic acid that hybridizes under high stringency conditions to a nucleic acid molecule comprising SEQ ID NO:2 having a G at position 1000 but not to a nucleic acid molecule comprising SEQ ID NO:2 having a T at position 1000;
(iiii) an oligonucleotide or peptide nucleic acid that hybridizes under high stringency conditions to a nucleic acid molecule comprising SEQ ID NO:2 having a G at position 1063 but not to a nucleic acid molecule comprising SEQ ID NO:2 having a C at position 1063;
(jjjj) an oligonucleotide or peptide nucleic acid that hybridizes under high stringency conditions to a nucleic acid molecule comprising SEQ ID NO:2 having a C at position 1063 but not to a nucleic acid molecule comprising SEQ ID NO:2 having a G at position 1063;
(kkkk) an oligonucleotide or peptide nucleic acid that hybridizes under high stringency conditions to a nucleic acid molecule comprising SEQ ID NO:2 having a G at position 1079 but not to a nucleic acid molecule comprising SEQ ID NO:2 having a A at position 1079;
(llll) an oligonucleotide or peptide nucleic acid that hybridizes under high stringency conditions to a nucleic acid molecule comprising SEQ ID NO:2 having a A at position 1079 but not to a nucleic acid molecule comprising SEQ ID NO:2 having a G at position 1079;
(mmmm) an oligonucleotide or peptide nucleic acid that hybridizes under high stringency conditions to a nucleic acid molecule comprising SEQ ID NO:2 having a C at position 1161 but not to a nucleic acid molecule comprising SEQ ID NO:2 having a A at position 1161;
(nnnn) an oligonucleotide or peptide nucleic acid that hybridizes under high stringency conditions to a nucleic acid molecule comprising SEQ ID NO:2 having a A at position 1161 but not to a nucleic acid molecule comprising SEQ ID NO:2 having a C at position 1161;
(oooo) an oligonucleotide or peptide nucleic acid that hybridizes under high stringency conditions to a nucleic acid molecule comprising SEQ ID NO:2 having a T at position 1162 but not to a nucleic acid molecule comprising SEQ ID NO:2 having a C at position 1162;
(pppp) an oligonucleotide or peptide nucleic acid that hybridizes under high stringency conditions to a nucleic acid molecule comprising SEQ ID NO:2 having a C at position 1162 but not to a nucleic acid molecule comprising SEQ ID NO:2 having a T at position 1162;
(qqqq) an oligonucleotide or peptide nucleic acid that hybridizes under high stringency conditions to a nucleic acid molecule comprising SEQ ID NO:2 having a C at position 1215 but not to a nucleic acid molecule comprising SEQ ID NO:2 having a T at position 1215; and
(rrrr) an oligonucleotide or peptide nucleic acid that hybridizes under high stringency conditions to a nucleic acid molecule comprising SEQ ID NO:2 having a T at position 1215 but not to a nucleic acid molecule comprising SEQ ID NO:2 having a C at position 1215.

38. An oligonucleotide or peptide nucleic acid selected from the group consisting of:
(a) an oligonucleotide or peptide nucleic acid that hybridizes under high stringency conditions to a nucleic acid molecule comprising SEQ ID NO:3 having a A at position 295 but not to a nucleic acid molecule comprising SEQ ID NO: 1 having a G at position 295;
(b) an oligonucleotide or peptide nucleic acid that hybridizes under high stringency conditions to a nucleic acid molecule comprising SEQ ID NO: 1 having a G at position 295 but not to a nucleic acid molecule comprising SEQ ID NO:3 having a A at position 295;
(c) an oligonucleotide or peptide nucleic acid that hybridizes under high stringency conditions to a nucleic acid molecule comprising SEQ ID NO:3 having a C at position 298 but not to a nucleic acid molecule comprising SEQ ID NO: 1 having a A at position 298;
(d) an oligonucleotide or peptide nucleic acid that hybridizes under high stringency conditions to a nucleic acid molecule comprising SEQ ID NO: 1 having a A at position 298 but not to a nucleic acid molecule comprising SEQ ID NO:3 having a C at position 298;
(e) an oligonucleotide or peptide nucleic acid that hybridizes under high stringency conditions to a nucleic acid molecule comprising SEQ ID NO: 1 having a A at position 301 but not to a nucleic acid molecule comprising SEQ ID NO:3 having a C at position 301;
(f) an oligonucleotide or peptide nucleic acid that hybridizes under high stringency conditions to a nucleic acid molecule comprising SEQ ID NO:3 having a C at position 301 but not to a nucleic acid molecule comprising SEQ ID NO: 1 having a A at position 301;
(g) an oligonucleotide or peptide nucleic acid that hybridizes under high stringency conditions to a nucleic acid molecule comprising SEQ ID NO:4 having a A at position 315 but not to a nucleic acid molecule comprising SEQ ID NO:1 having a G at position 315;
(h) an oligonucleotide or peptide nucleic acid that hybridizes under high stringency conditions to a nucleic acid molecule comprising SEQ ID NO: 1 having a G at position 315 but not to a nucleic acid molecule comprising SEQ ID NO:4 having a A at position 315;
(i) an oligonucleotide or peptide nucleic acid that hybridizes under high stringency conditions to a nucleic acid molecule comprising SEQ ID NO: 1 having a C at position 318 but not to a nucleic acid molecule comprising SEQ ID NO:4 having a A at position 318;
(j) an oligonucleotide or peptide nucleic acid that hybridizes under high stringency conditions to a nucleic acid molecule comprising SEQ ID NO:4 having a A at position 318 but not to a nucleic acid molecule comprising SEQ ID NO:1 having a C at position 318;
(k) an oligonucleotide or peptide nucleic acid that hybridizes under high stringency conditions to a nucleic acid molecule comprising SEQ ID NO: 1 having a A at position 319 but not to a nucleic acid molecule comprising SEQ ID NO:4 having a C at position 319;
(l) an oligonucleotide or peptide nucleic acid that hybridizes under high stringency conditions to a nucleic acid molecule comprising SEQ ID NO:4 having a C at position 319 but not to a nucleic acid molecule comprising SEQ ID NO: 1 having a A at position 319;
(m) an oligonucleotide or peptide nucleic acid that hybridizes under high stringency conditions to a nucleic acid molecule comprising SEQ ID NO:5 having a A at position 734 but not to a nucleic acid molecule comprising SEQ ID NO: 1 having a G at position 734;
(n) an oligonucleotide or peptide nucleic acid that hybridizes under high stringency conditions to a nucleic acid molecule comprising SEQ ID NO: 1 having a G at position 734 but not to a nucleic acid molecule comprising SEQ ID NO:5 having a A at position 734;
(o) an oligonucleotide or peptide nucleic acid that hybridizes under high stringency conditions to a nucleic acid molecule comprising SEQ ID NO:5 having a C at position 735 but not to a nucleic acid molecule comprising SEQ ID NO: 1 having a A at position 735;
(p) an oligonucleotide or peptide nucleic acid that hybridizes under high stringency conditions to a nucleic acid molecule comprising SEQ ID NO: 1 having a A at position 735 but not to a nucleic acid molecule comprising SEQ ID NO:5 having a C at position 735;
(q) an oligonucleotide or peptide nucleic acid that hybridizes under high stringency conditions to a nucleic acid molecule comprising SEQ ID NO:6 having a T at position 934 but not to a nucleic acid molecule comprising SEQ ID NO: 1 having a C at position 934;
(r) an oligonucleotide or peptide nucleic acid that hybridizes under high stringency conditions to a nucleic acid molecule comprising SEQ ID NO: 1 having a C at position 934 but not to a nucleic acid molecule comprising SEQ ID NO:6 having a T at position 934;
(s) an oligonucleotide or peptide nucleic acid that hybridizes under high stringency conditions to a nucleic acid molecule comprising SEQ ID NO:6 having a G at position 935 but not to a nucleic acid molecule comprising SEQ ID NO: 1 having a T at position 935;
(t) an oligonucleotide or peptide nucleic acid that hybridizes under high stringency conditions to a nucleic acid molecule comprising SEQ ID NO: 1 having a T at position 935 but not to a nucleic acid molecule comprising SEQ ID NO:6 having a G at position 935;
(u) an oligonucleotide or peptide nucleic acid that hybridizes under high stringency conditions to a nucleic acid molecule comprising SEQ ID NO:7 having a G at position 1085 but not to a nucleic acid molecule comprising SEQ ID NO: 1 having a C at position 1085;
(v) an oligonucleotide or peptide nucleic acid that hybridizes under high stringency conditions to a nucleic acid molecule comprising SEQ ID NO: 1 having a C at position 1085 but not to a nucleic acid molecule comprising SEQ ID NO:7 having a G at position 1085;
(w) an oligonucleotide or peptide nucleic acid that hybridizes under high stringency conditions to a nucleic acid molecule comprising SEQ ID NO:7 having a C at position 1087 but not to a nucleic acid molecule comprising SEQ ID NO: 1 having a G at position 1087;
(x) an oligonucleotide or peptide nucleic acid that hybridizes under high stringency conditions to a nucleic acid molecule comprising SEQ ID NO: 1 having a G at position 1087 but not to a nucleic acid molecule comprising SEQ ID NO:7 having a C at position 1087;
(y) an oligonucleotide or peptide nucleic acid that hybridizes under high stringency conditions to a nucleic acid molecule comprising SEQ ID NO:7 having a C at position 1088 but not to a nucleic acid molecule comprising SEQ ID NO: 1 having a G at position 1088;
(z) an oligonucleotide or peptide nucleic acid that hybridizes under high stringency conditions to a nucleic acid molecule comprising SEQ ID NO: 1 having a G at position 1088 but not to a nucleic acid molecule comprising SEQ ID NO:7 having a C at position 1088;
(aa) an oligonucleotide or peptide nucleic acid that hybridizes under high stringency conditions to a nucleic acid molecule comprising SEQ ID NO:7 having a T at position 1089 but not to a nucleic acid molecule comprising SEQ ID NO: 1 having a G at position 1089;
(bb) an oligonucleotide or peptide nucleic acid that hybridizes under high stringency conditions to a nucleic acid molecule comprising SEQ ID NO: 1 having a G at position 1089 but not to a nucleic acid molecule comprising SEQ ID NO:7 having a T at position 1089;
(cc) an oligonucleotide or peptide nucleic acid that hybridizes under high stringency conditions to a nucleic acid molecule comprising SEQ ID NO:7 having a G at position 1090 but not to a nucleic acid molecule comprising SEQ ID NO: 1 having a T at position 1090;
(dd) an oligonucleotide or peptide nucleic acid that hybridizes under high stringency conditions to a nucleic acid molecule comprising SEQ ID NO: 1 having a T at position 1090 but not to a nucleic acid molecule comprising SEQ ID NO:7 having a G at position 1090;
(ee) an oligonucleotide or peptide nucleic acid that hybridizes under high stringency conditions to a nucleic acid molecule comprising SEQ ID NO:7 having a G at position 1091 but not to a nucleic acid molecule comprising SEQ ID NO:1 having a T at position 1091;
(ff) an oligonucleotide or peptide nucleic acid that hybridizes under high stringency conditions to a nucleic acid molecule comprising SEQ ID NO: 1 having a T at position 1091 but not to a nucleic acid molecule comprising SEQ ID NO:7 having a G at position 1091;
(gg) an oligonucleotide or peptide nucleic acid that hybridizes under high stringency conditions to a nucleic acid molecule comprising SEQ ID NO:7 having a A at position 1092 but not to a nucleic acid molecule comprising SEQ ID NO: 1 having a G at position 1092;
(hh) an oligonucleotide or peptide nucleic acid that hybridizes under high stringency conditions to a nucleic acid molecule comprising SEQ ID NO: 1 having a G at position 1092 but not to a nucleic acid molecule comprising SEQ ID NO:7 having a A at position 1092;
(ii) an oligonucleotide or peptide nucleic acid that hybridizes under high stringency conditions to a nucleic acid molecule comprising SEQ ID NO:7 having a C at position 1093 but not to a nucleic acid molecule comprising SEQ ID NO: 1 having a G at position 1093;
(jj) an oligonucleotide or peptide nucleic acid that hybridizes under high stringency conditions to a nucleic acid molecule comprising SEQ ID NO: 1 having a G at position 1093 but not to a nucleic acid molecule comprising SEQ ID NO:7 having a C at position 1093;
(kk) an oligonucleotide or peptide nucleic acid that hybridizes under high stringency conditions to a nucleic acid molecule comprising SEQ ID NO: 8 having a A at position 1161 but not to a nucleic acid molecule comprising SEQ ID NO: 1 having a C at position 1161;
(ll) an oligonucleotide or peptide nucleic acid that hybridizes under high stringency conditions to a nucleic acid molecule comprising SEQ ID NO: 1 having a C at position 1161 but not to a nucleic acid molecule comprising SEQ ID NO:8 having a A at position 1161;
(mm) an oligonucleotide or peptide nucleic acid that hybridizes under high stringency conditions to a nucleic acid molecule comprising SEQ ID NO: 8 having a C at position 1162 but not to a nucleic acid molecule comprising SEQ ID NO: 1 having a T at position 1162;
(nn) an oligonucleotide or peptide nucleic acid that hybridizes under high stringency conditions to a nucleic acid molecule comprising SEQ ID NO: 1 having a T at position 1162 but not to a nucleic acid molecule comprising SEQ ID NO:8 having a C at position 1162;
(oo) an oligonucleotide or peptide nucleic acid that hybridizes under high stringency conditions to a nucleic acid molecule comprising SEQ ID NO: 8 having a G at position 1163 but not to a nucleic acid molecule comprising SEQ ID NO: 1 having a A at position 1163;
(pp) an oligonucleotide or peptide nucleic acid that hybridizes under high stringency conditions to a nucleic acid molecule comprising SEQ ID NO: 1 having a A at position 1163 but not to a nucleic acid molecule comprising SEQ ID NO:8 having a G at position 1163;
(qq) an oligonucleotide or peptide nucleic acid that hybridizes under high stringency conditions to a nucleic acid molecule comprising SEQ ID NO: 8 having a G at position 1164 but not to a nucleic acid molecule comprising SEQ ID NO:1 having a T at position 1164;
(rr) an oligonucleotide or peptide nucleic acid that hybridizes under high stringency conditions to a nucleic acid molecule comprising SEQ ID NO: 1 having a T at position 1164 but not to a nucleic acid molecule comprising SEQ ID NO:8 having a G at position 1164;
(ss) an oligonucleotide or peptide nucleic acid that hybridizes under high stringency conditions to a nucleic acid molecule comprising SEQ ID NO: 10 having a T at position 1215 but not to a nucleic acid molecule comprising SEQ ID NO:1 having a C at position 1215;
(tt) an oligonucleotide or peptide nucleic acid that hybridizes under high stringency conditions to a nucleic acid molecule comprising SEQ ID NO: 1 having a C at position 1215 but not to a nucleic acid molecule comprising SEQ ID NO:10 having a T at position 1215;
(uu) an oligonucleotide or peptide nucleic acid that hybridizes under high stringency conditions to a nucleic acid molecule comprising SEQ ID NO: 10 having a T at position 1216 but not to a nucleic acid molecule comprising SEQ ID NO:1 having a G at position 1216;
(vv) an oligonucleotide or peptide nucleic acid that hybridizes under high stringency conditions to a nucleic acid molecule comprising SEQ ID NO: 1 having a G at position 1216 but not to a nucleic acid molecule comprising SEQ ID NO:10 having a T at position 1216;
(ww) an oligonucleotide or peptide nucleic acid that hybridizes under high stringency conditions to a nucleic acid molecule comprising SEQ ID NO: 10 having a A at position 1217 but not to a nucleic acid molecule comprising SEQ ID NO:1 having a G at position 1217;
(xx) an oligonucleotide or peptide nucleic acid that hybridizes under high stringency conditions to a nucleic acid molecule comprising SEQ ID NO: 1 having a G at position 1217 but not to a nucleic acid molecule comprising SEQ ID NO:10 having a A at position 1217;
(yy) an oligonucleotide or peptide nucleic acid that hybridizes under high stringency conditions to a nucleic acid molecule comprising SEQ ID NO: 10 having a C at position 1219 but not to a nucleic acid molecule comprising SEQ ID NO: 1 having a G at position 1219;
(zz) an oligonucleotide or peptide nucleic acid that hybridizes under high stringency conditions to a nucleic acid molecule comprising SEQ ID NO: 1 having a G at position 1219 but not to a nucleic acid molecule comprising SEQ ID NO: 10 having a C at position 1219;
(aaa) an oligonucleotide or peptide nucleic acid that hybridizes under high stringency conditions to a nucleic acid molecule comprising SEQ ID NO: 10 having a C at position 1220 but not to a nucleic acid molecule comprising SEQ ID NO: 1 having a T at position 1220;
(bbb) an oligonucleotide or peptide nucleic acid that hybridizes under high stringency conditions to a nucleic acid molecule comprising SEQ ID NO: 1 having a T at position 1220 but not to a nucleic acid molecule comprising SEQ ID NO: 10 having a C at position 1220;
(ccc) an oligonucleotide or peptide nucleic acid that hybridizes under high stringency conditions to a nucleic acid molecule comprising SEQ ID NO: 10 having a C at position 1221 but not to a nucleic acid molecule comprising SEQ ID NO: 1 having a G at position 1221;
(ddd) an oligonucleotide or peptide nucleic acid that hybridizes under high stringency conditions to a nucleic acid molecule comprising SEQ ID NO: 1 having a G at position 1221 but not to a nucleic acid molecule comprising SEQ ID NO:10 having a C at position 1221;
(eee) an oligonucleotide or peptide nucleic acid that hybridizes under high stringency conditions to a nucleic acid molecule comprising SEQ ID NO: 10 having a A at position 1222 but not to a nucleic acid molecule comprising SEQ ID NO: 1 having a G at position 1222;
(fff) an oligonucleotide or peptide nucleic acid that hybridizes under high stringency conditions to a nucleic acid molecule comprising SEQ ID NO: 1 having a G at position 1222 but not to a nucleic acid molecule comprising SEQ ID NO: 10 having a A at position 1222;
(ggg) an oligonucleotide or peptide nucleic acid that hybridizes under high stringency conditions to a nucleic acid molecule comprising SEQ ID NO: 10 having a G at position 1223 but not to a nucleic acid molecule comprising SEQ ID NO: 1 having a A at position 1223;
(hhh) an oligonucleotide or peptide nucleic acid that hybridizes under high stringency conditions to a nucleic acid molecule comprising SEQ ID NO: 1 having a A at position 1223 but not to a nucleic acid molecule comprising SEQ ID NO: 10 having a G at position 1223;
(iii) an oligonucleotide or peptide nucleic acid that hybridizes under high stringency conditions to a nucleic acid molecule comprising SEQ ID NO: 10 having a A at position 1224 but not to a nucleic acid molecule comprising SEQ ID NO: 1 having a G at position 1224;
(jjj) an oligonucleotide or peptide nucleic acid that hybridizes under high stringency conditions to a nucleic acid molecule comprising SEQ ID NO: 1 having a G at position 1224 but not to a nucleic acid molecule comprising SEQ ID NO: 10 having a A at position 1224;
(kkk) an oligonucleotide or peptide nucleic acid that hybridizes under high stringency conditions to a nucleic acid molecule comprising SEQ ID NO: 10 having a C at position 1226 but not to a nucleic acid molecule comprising SEQ ID NO: 1 having a G at position 1226;
(lll) an oligonucleotide or peptide nucleic acid that hybridizes under high stringency conditions to a nucleic acid molecule comprising SEQ ID NO: 1 having a G at position 1226 but not to a nucleic acid molecule comprising SEQ ID NO: 10 having a C at position 1226;
(mmm) an oligonucleotide or peptide nucleic acid that hybridizes under high stringency conditions to a nucleic acid molecule comprising SEQ ID NO: 10 having a T at position 1227 but not to a nucleic acid molecule comprising SEQ ID NO: 1 having a G at position 1227;
(nnn) an oligonucleotide or peptide nucleic acid that hybridizes under high stringency conditions to a nucleic acid molecule comprising SEQ ID NO: 1 having a G at position 1227 but not to a nucleic acid molecule comprising SEQ ID NO: 10 having a T at position 1227;
(ooo) an oligonucleotide or peptide nucleic acid that hybridizes under high stringency conditions to a nucleic acid molecule comprising SEQ ID NO: 10 having a C at position 1228 but not to a nucleic acid molecule comprising SEQ ID NO: 1 having a A at position 1228;
(ppp) an oligonucleotide or peptide nucleic acid that hybridizes under high stringency conditions to a nucleic acid molecule comprising SEQ ID NO: 1 having a A at position 1228 but not to a nucleic acid molecule comprising SEQ ID NO: 10 having a C at position 1228;
(qqq) an oligonucleotide or peptide nucleic acid that hybridizes under high stringency conditions to a nucleic acid molecule comprising SEQ ID NO: 10 having a T at position 1229 but not to a nucleic acid molecule comprising SEQ ID NO: 1 having a G at position 1229;
(rrr) an oligonucleotide or peptide nucleic acid that hybridizes under high stringency conditions to a nucleic acid molecule comprising SEQ ID NO: 1 having a G at position 1229 but not to a nucleic acid molecule comprising SEQ ID NO: 10 having a T at position 1229;
(sss) an oligonucleotide or peptide nucleic acid that hybridizes under high stringency conditions to a nucleic acid molecule comprising SEQ ID NO: 10 having a G at position 1230 but not to a nucleic acid molecule comprising SEQ ID NO: 1 having a A at position 1230;
(ttt) an oligonucleotide or peptide nucleic acid that hybridizes under high stringency conditions to a nucleic acid molecule comprising SEQ ID NO: 1 having a A at position 1230 but not to a nucleic acid molecule comprising SEQ ID NO: 10 having a G at position 1230;
(uuu) an oligonucleotide or peptide nucleic acid that hybridizes under high stringency conditions to a nucleic acid molecule comprising SEQ ID NO: 10 having a A at position 1231 but not to a nucleic acid molecule comprising SEQ ID NO:1 having a T at position 1231;
(vvv) an oligonucleotide or peptide nucleic acid that hybridizes under high stringency conditions to a nucleic acid molecule comprising SEQ ID NO: 1 having a T at position 1231 but not to a nucleic acid molecule comprising SEQ ID NO: 10 having a A at position 1231;
(www) an oligonucleotide or peptide nucleic acid that hybridizes under high stringency conditions to a nucleic acid molecule comprising SEQ ID NO: 10 having a G at position 1232 but not to a nucleic acid molecule comprising SEQ ID NO: 1 having a A at position 1232;
(xxx) an oligonucleotide or peptide nucleic acid that hybridizes under high stringency conditions to a nucleic acid molecule comprising SEQ ID NO: 1 having a A at position 1232 but not to a nucleic acid molecule comprising SEQ ID NO: 10 having a G at position 1232;
(yyy) an oligonucleotide or peptide nucleic acid that hybridizes under high stringency conditions to a nucleic acid molecule comprising SEQ ID NO: 10 having a T at position 1233 but not to a nucleic acid molecule comprising SEQ ID NO: 1 having a G at position 1233;
(zzz) an oligonucleotide or peptide nucleic acid that hybridizes under high stringency conditions to a nucleic acid molecule comprising SEQ ID NO: 1 having a G at position 1233 but not to a nucleic acid molecule comprising SEQ ID NO: 10 having a T at position 1233;
(aaaa) an oligonucleotide or peptide nucleic acid that hybridizes under high stringency conditions to a nucleic acid molecule comprising SEQ ID NO: 10 having a G at position 1234 but not to a nucleic acid molecule comprising SEQ ID NO: 1 having a A at position 1234;
(bbbb) an oligonucleotide or peptide nucleic acid that hybridizes under high stringency conditions to a nucleic acid molecule comprising SEQ ID NO: 1 having a A at position 1234 but not to a nucleic acid molecule comprising SEQ ID NO: 10 having a G at position 1234;
(cccc) an oligonucleotide or peptide nucleic acid that hybridizes under high stringency conditions to a nucleic acid molecule comprising SEQ ID NO: 10 having a G at position 1235 but not to a nucleic acid molecule comprising SEQ ID NO: 1 having a C at position 1235;
(dddd) an oligonucleotide or peptide nucleic acid that hybridizes under high stringency conditions to a nucleic acid molecule comprising SEQ ID NO: 1 having a C at position 1235 but not to a nucleic acid molecule comprising SEQ ID NO: 10 having a G at position 1235;
(eeee) an oligonucleotide or peptide nucleic acid that hybridizes under high stringency conditions to a nucleic acid molecule comprising SEQ ID NO: 10 having a T at position 1236 but not to a nucleic acid molecule comprising SEQ ID NO: 1 having a C at position 1236;
(ffff) an oligonucleotide or peptide nucleic acid that hybridizes under high stringency conditions to a nucleic acid molecule comprising SEQ ID NO: 1 having a C at position 1236 but not to a nucleic acid molecule comprising SEQ ID NO: 10 having a T at position 1236;
(gggg) an oligonucleotide or peptide nucleic acid that hybridizes under high stringency conditions to a nucleic acid molecule comprising SEQ ID NO: 10 having a T at position 1237 but not to a nucleic acid molecule comprising SEQ ID NO: 1 having a C at position 1237;
(hhhh) an oligonucleotide or peptide nucleic acid that hybridizes under high stringency conditions to a nucleic acid molecule comprising SEQ ID NO: 1 having a C at position 1237 but not to a nucleic acid molecule comprising SEQ ID NO: 10 having a T at position 1237;
(iiii) an oligonucleotide or peptide nucleic acid that hybridizes under high stringency conditions to a nucleic acid molecule comprising SEQ ID NO: 10 having a T at position 1238 but not to a nucleic acid molecule comprising SEQ ID NO: 1 having a A at position 1238;
(jjjj) an oligonucleotide or peptide nucleic acid that hybridizes under high stringency conditions to a nucleic acid molecule comprising SEQ ID NO: 1 having a A at position 1238 but not to a nucleic acid molecule comprising SEQ ID NO: 10 having a T at position 1238;
(kkkk) an oligonucleotide or peptide nucleic acid that hybridizes under high stringency conditions to a nucleic acid molecule comprising SEQ ID NO: 10 having a C at position 1239 but not to a nucleic acid molecule comprising SEQ ID NO: 1 having a G at position 1239; and
(llll) an oligonucleotide or peptide nucleic acid that hybridizes under high stringency conditions to a nucleic acid molecule comprising SEQ ID NO: 1 having a G at position 1239 but not to a nucleic acid molecule comprising SEQ ID NO: 10 having a C at position 1239.

39. An array of nucleic acid molecules attached to a solid support, the array comprising one or more oligonucleotide or peptide nucleic acids or peptide nucleic acids of claim 37.

40. The oligonucleotide or peptide nucleic acid of claim 37, further comprising one or more of the following: a detectable label; a quencher; a mobility modifier; a contiguous non-target sequence situated 5′ or 3′ to the target sequence or 5′ and 3′ to the target sequence.

41. A computer readable medium comprising a plurality of digitally encoded mutational correlations selected from the SPARC promoter correlations in TABLE 4, wherein each correlation of the plurality has a value representing a sensitivity to a therapeutic regimen.

42. A method of detecting a mutation in a SPARC promoter occurring in an animal, wherein the mutation results in reduced intracellular and/or extracellular levels of a polypeptide encoded by the SPARC gene, comprising:
(a) determining the nucleic acid sequence of the SPARC promoter, and
(b) comparing the determined the SPARC promoter nucleic acid sequence to a database of wild-type nucleic acid sequences which correspond to the determined SPARC promoter nucleic acid sequence.

43. The method of claim 42, further comprising obtaining nucleic acids from the animal.

44. The method of claim 42, wherein the nucleic acid is amplified prior to determining the nucleic acid sequence of SPARC promoter nucleic acid.

45. The method of claim 44, wherein the presence of a mutation is indicative of a cancer in the animal having resistance to a therapeutic regimen.

46. The method of claim 45, wherein the cancer is selected from the following: carcinoma; melanoma; sarcoma; lymphoma; and leukaemia.

47. The method of claim 46, wherein the carcinoma is selected from the following: squamous cell; adeno; small cell; large cell and combinations thereof.

48. The method of claim 46, wherein the carcinoma is derived from a primary tumor located in nasopharygeal tissue, head and neck, lung, esophagus, stomach, intestine, rectum, kidney, urinary tract, prostate, testes, ovary, uterus, cervix, vagina, skin, and endocrine glands.

49. The method of claim 46, wherein the sarcoma is selected from the following: muscle; adipose; fibrous; vascular; and neural.

50. The method of claim 46, wherein the lymphoma is selected from the following: Hodgkin's Disease; and Non-Hodgkins Lymphoma.

51. The method of claim 46, wherein the leukaemia is selected from the following: lymphoid; and myeloid.

52. The method of claim 45, wherein the cancer is a colorectal carcinoma, prostate carcinoma, ovarian carcinoma or osteosarcoma.

53. The method of claim 43, wherein the presence of the mutation further indicates resistance to chemotherapy, radiation therapy or combinations thereof.

54. The method of claim 53, wherein the resistance is to 5-flurouracil, irinotecan, cisplatin, doxorubicin or combinations thereof.

55. The method of claim 42, wherein the mutation is in a transcriptional control sequence of the SPARC promoter.

56. The method of claim 55, wherein the control sequence is a promoter, enhancer, repressor or polyadenylation control sequence.

57. The method of claim 56, wherein the SPARC promoter is represented by one of SEQ ID NOS: 1-11.

58. The method of claim 42, wherein the mutation is a point mutation, deletion, insertion or combination thereof.

59. The method of claim 42, wherein the animal is a human.

60. The method of claim 44, wherein the nucleic acid amplified is DNA or RNA.

61. The method of claim 44, wherein the nucleic acid amplification is done by linear amplification, Reverse Transcription Polymerase Chain Reaction (RT-PCR), Polymerase Chain Reaction (PCR), Transcription Mediated Amplification (TMA), Nucleic Acid Sequence Based Amplification (NASBA) or combinations thereof.

62. The method of claim 44, wherein determination of the SPARC promoter sequence is done by hybridization, Restriction Fragment Length Polymorphism (RFLP) assay, Single-Stranded Conformational Polymorphism (SSCP) assay or a Cleavase Fragment Length Polymorphism (CFLP) assay, RNAase protection, chain termination sequencing, chemical sequencing, mass spectroscopy or combinations thereof.

63. The method of claim 42, wherein the determination of the SPARC promoter sequence is done by hybridization, RFLP assay, Invader® assay, Ligase chain reaction (LCR), oligonucleotide or peptide nucleic acid ligation assay, ligation-rolling circle amplification, allele specific PCR, single base-pair extension assays or combinations thereof.

64. The method of claim 42, wherein the SPARC promoter sequence determined comprises SEQ ID NO: 1.

65. The method of claim 42, wherein the SPARC promoter sequence determined comprises SEQ ID NOS:3-33.

66. The method of claim 42, wherein the SPARC promoter sequence determined comprises SEQ ID NO: 2.

67. The method of claim 44, wherein the amplification is done by PCR using the primer pairs represented by one or more of SEQ ID NO:34 and SEQ ID NO:35, SEQ ID NO:36 and SEQ ID NO 37, SEQ ID NO:38 and SEQ ID NO:39 or combinations thereof.

68. The method of claim 42, wherein the mutation detected is one or more of the following mutations relative to SEQ ID NO: 1:
(a) 295delG and 301A>CC;
(b) 315insA and 316G>A;
(c) 341C>T, 342T>C, 344A>C, 347C>A, 354G>A, 356G>A, 359T>G and 363G>A;
(d) 395C>T and 396A>T;
(e) 488T>C;
(f) 533A>G;
(g) 734G>A;
(h) 734delG;
(i) 769T>A, 771G>C and 774G>C;
(j) 784T>C, 786T>G and 788T>A;
(k) 793T>G, 796T>A and 800T>C;
(l) 820C>A and 824T>A;
(m) 864T>A and 867T>A;
(n) 895T>G, 899T>A, 901G>A and 904G>A;
(o) 901G>A;
(p) 901G>A, 904G>A, 929A>G and 934delC;
(q) 922T>G, 928T>G and 935T>G;
(r) 982T>C, 984T>G, 985G>A, 991G>A, 995G>A, 999C>T and 1000T>G;
(s) 1063G>C;
(t) 1079G>A;
(u) 1085delC, 1086delA, 1087delG, 1089G>A, 1090T>C, 1091T>C and 1092G>T;
(v) 1161C>A, 1162T>C, 1163delA and 1164delT; and
(w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T.

69. A method of treating a cancer in a subject in need thereof, the method comprising administering to the subject a therapeutic regimen, wherein said subject has a SPARC promoter genotype indicative of sensitivity to the therapeutic regimen.

70. A method of treating a cancer in a subject in need thereof, the method comprising:
(a) selecting a subject having a SPARC promoter genotype indicative of sensitivity to a therapeutic regimen; and
(b) administering to said subject the therapeutic regimen.

71. A method of treating a cancer in a subject in need thereof, the method comprising:
(a) selecting a subject having a resistant genotype in a SPARC promoter sequence; and
(b) administering to said subject an alternative therapeutic regimen.

72. A method of identifying a subject with an increased sensitivity to a therapeutic regimen for treating a cancer in a subject in need thereof, comprising the step of screening a population of subjects to identify those subjects that have a mutation in their SPARC promoter sequence, wherein the identification of a subject with a mutation is predictive of decreased responsiveness to the therapeutic regimen.

73. A method of treating a cancer in a subject in need thereof, the method comprising administering a therapeutic regimen to the subject, wherein said subject does not have a mutation indicative of resistance to the therapeutic regimen in their SPARC promoter sequence.

74. The method of claim 69, wherein the SPARC promoter mutation, is selected from one or more of the following mutations relative to SEQ ID NO:1:
(a) 295delG and 301A>CC;
(b) 315insA and 316G>A;
(c) 341C>T, 342T>C, 344A>C, 347C>A, 354G>A, 356G>A, 359T>G and 363G>A;
(d) 395C>T and 396A>T;
(e) 488T>C;
(f) 533A>G;
(g) 734G>A;
(h) 734delG;
(i) 769T>A, 771G>C and 774G>C;
(j) 784T>C, 786T>G and 788T>A;
(k) 793T>G, 796T>A and 800T>C;
(l) 820C>A and 824T>A;
(m) 864T>A and 867T>A;
(n) 895T>G, 899T>A, 901G>A and 904G>A;
(o) 901G>A;
(p) 901G>A, 904G>A, 929A>G and 934delC;
(q) 922T>G, 928T>G and 935T>G;
(r) 982T>C, 984T>G, 985G>A, 991G>A, 995G>A, 999C>T and 1OOOT>G;
(s) 1063G>C;
(t) 1079G>A;
(u) 1085delC, 1086delA, 1087delG, 1089G>A, 1090T>C, 1091T>C and 1092G>T;
(v) 1161C>A, 1162T>C, 1163delA and 1164delT; and
(w) 1215C>T, 1216delG, 1217delG, 1218delG, 1219delG, 1220delT, 1221delG, 1222delG, 1223delA, 1224delG, 1225delG, 1226delG, 1227delG, 1228delA, 1229delG, 1230delA, 1231delT, 1232delA, 1233delG, 1234delA, 1235delC, 1236delC and 1237C>T.

75. The method of claim 69, wherein the cancer is selected from the following: carcinoma; melanoma; sarcoma; lymphoma; and leukaemia.

76. The method of claim 75, wherein the carcinoma is selected from the following: squamous cell; adeno; small cell; large cell and combinations thereof.

77. The method of claim 76, wherein the carcinoma is derived from a primary tumor located in nasopharygeal tissue, head and neck, lung, esophagus, stomach, intestine, rectum, kidney, urinary tract, prostate, testes, ovary, uterus, cervix, vagina, skin, and endocrine glands.

78. The method of claim 75, wherein the sarcoma is selected from the following: muscle; adipose; fibrous; vascular; and neural.

79. The method of claim 75, wherein the lymphoma is selected from the following: Hodgkin's Disease; and Non-Hodgkins Lymphoma.

80. The method of claim 75, wherein the leukaemia is selected from the following: lymphoid; and myeloid.

81. The method of claim 75, wherein the cancer is a colorectal carcinoma, prostate carcinoma, ovarian carcinoma or osteosarcoma.

82. The method of claim 69, wherein the cancer is a SPARC inhibited cancer.

83-94. (canceled)


SEQUENCE LISTING (55 sequences)

SEQ ID NO: 1 - DNA, 1370 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 2 - DNA, 1370 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccgraamccmcaaaactttttgaggacaaggaccagatatttattaattyycmtamctcccarartgktagrcacgaaataaacattcaaccaagacctgttgywctgagcagttcatatataacaggagtgacccaagttgaaacgtagaatcagccctctcataccactttttggcaggtgatcataggcaagtyacttagcatctatgtttccttattattaaaatggtcataattacratgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaaraccagttccaaatcatcaaggatgataccactagwascascttgtcttgyckgwacagkggwaagycctggccttgcctttgtggmaaawacaacccccttgaattgcttggcccttctcagcattgccwaawattagggaggactcctgtaaagctcackggtwaraaratcaagacacttgggcckggttckrccccckgggggccattgggtaattccttgcagtctccaggcctcacttgcccyckraacaaraaaraggykgttctgggtcatccctccaggcctgtccagccctggcactctgtgagtcggtttaggcagcasccccggaacagatgargcaggcagggttgggacgtttggtcaggacagcccaccgcaccaagaggaggaaagaaatgaaagacagagacagctttggmyatgggagaaggaggaggccgggggaaggaggagacaggaggaggagggaccayggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 3 - DNA, 1370 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccgaaacccccaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccccctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 4 - DNA, 1371 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaagaacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccccctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 5 - DNA, 1369 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaaaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccccctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 6 - DNA, 1369 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttaaaaaatcaagacacttgggcctggttctgcccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 7 - DNA, 1367 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcagggacctggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 8 - DNA, 1368 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggacgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 9 - DNA, 2963 bp, Homo sapiens
gattgaattccttgtactttttttcccttctcagttctgcacttaactcgtctaaaaaaattaaaaaagaatttaagaaaccacaaagctaagctgggtgcggtggctcacgcctgtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccaggtatttattaattctcatacctcccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcaattaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctgcccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagttaagaaaccacaaagctaagctgggtgcggtggctcacgcctgtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccaggtatttattaattctcatacctcccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcaattaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctgcccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgaggtatgtgtgacccccgcccagcctttcccttctatagttgcaccaaccccgacacccccgttcacgccgtcagctcgtgtgcaagggaggg

SEQ ID NO: 10 - DNA, 1349 bp, Homo sapiens
aatcccagcactttgggaggccgaggtgggtggatcatgaggtcaggcattcgagaccagcctggccaacatagtgaaaccccatctctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccattagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 11 - DNA, 399 bp, Homo sapiens
aataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccgaaacccccaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatct

SEQ ID NO: 12 - DNA, 407 bp, Homo sapiens
gtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaagaacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaat

SEQ ID NO: 13 - DNA, 400 bp, Homo sapiens
ccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaatttccctaactcccaaaatggtagacacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacaatgccta

SEQ ID NO: 14 - DNA, 406 bp, Homo sapiens
ttgcagtgagccgaaatcataccactgcactccagcctgggcgacagagtgagtgagactctgtctcaaaacaaaacaaaacaaacaaacaaaaaaaccggaaaccacaaaactttttgaggacaaggaccagatatttattaattctcatacctcccagagtgttaggcacgaaataaacattcaaccaagacctgttgttctgagcagttcatatataacaggagtgacccaagttgaaacgtagaatcagccctctcataccactttttggcaggtgatcataggcaagttacttagcatctatgtttccttattattaaaatggtcataattacaatgcctaagataaggggttgctgtgaagattattaaatcctcagtaaactttggctattgttactcc

SEQ ID NO: 15 - DNA, 403 bp, Homo sapiens
aaaaccggaaaccacaaaactttttgaggacaaggaccagatatttattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagtcacttagcatctatgtttccttattattaaaatggtcataattacaatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtct

SEQ ID NO: 16 - DNA, 408 bp, Homo sapiens
tattaattctcatacctcccagagtgttaggcacgaaataaacattcaaccaagacctgttgcactgagcagttcatatataacaggagtgacccaagttgaaacgtagaatcagccctctcataccactttttggcaggtgatcataggcaagttacttagcatctatgtttccttattattaaaatggtcataattacgatgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagt

SEQ ID NO: 17 - DNA, 407 bp, Homo sapiens
atgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaaaaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctggggg

SEQ ID NO: 18 - DNA, 406 bp, Homo sapiens
atgcctaagataaggggttgctgtgaagattattaaatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaaaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctggggg

SEQ ID NO: 19 - DNA, 402 bp, Homo sapiens
aatcctcagtaaactttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagaaccaccttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggc

SEQ ID NO: 20 - DNA, 407 bp, Homo sapiens
ttggctattgttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgccggaacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaa

SEQ ID NO: 21 - DNA, 398 bp, Homo sapiens
gttactcctatgattatcatcaatatcatcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacaggggaaagccctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaa

SEQ ID NO: 22 - DNA, 401 bp, Homo sapiens
atcacttaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggaaaaaacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccag

SEQ ID NO: 23 - DNA, 397 bp, Homo sapiens
gctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgccaaaaattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcag

SEQ ID NO: 24 - DNA, 446 bp, Homo sapiens
gtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcacgggtaaaaaaatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatg

SEQ ID NO: 25 - DNA, 440 bp, Homo sapiens
caagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttaaaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatg

SEQ ID NO: 26 - DNA, 439 bp, Homo sapiens
caagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttaaaaaatcaagacacttgggcctggttctgcccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatg

SEQ ID NO: 27 - DNA, 419 bp, Homo sapiens
ttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggccgggttcgacccccggggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatg

SEQ ID NO: 28 - DNA, 419 bp, Homo sapiens
tgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctaccccctgggggccattgggtaattccttgcagtctccaggcctcacttgcccccgaaacaaaaaaaaggtggttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacagg

SEQ ID NO: 29 - DNA, 418 bp, Homo sapiens
ctaatattagggaggactcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcacccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacc

SEQ ID NO: 30 - DNA, 402 bp, Homo sapiens
ctcctgtaaagctcactggttagaagatcaagacacttgggcctggttctaccccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaagcaggcagggttgggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacc

SEQ ID NO: 31 - DNA, 415 bp, Homo sapiens
aaagctcactggttagaagatcaagacacttgggcctggttctaccccctgggggccattgggtaattccttgcagtctccaggcctcacttgccccctgaacaagaaagaggctgttctgggtcatccctccaggcctgtccagccctggcactctgtgagtcggtttaggcagcagccccggaacagatgaggcagggacctggacgtttggtcaggacagcccaccgcaccaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttc

SEQ ID NO: 32 - DNA, 408 bp, Homo sapiens
gtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggacgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 33 - DNA, 339 bp, Homo sapiens
atccctccaggcctgtccagccctggcactctgtgagtcggtttaggcagcagccccggaacagatgaggcaggcagggttgggacgtttggtcaggacagcccaccgcaccaagaggaggaaagaaatgaaagacagagacagctttggctatgggagaaggaggaggccgggggaaggaggagacaggaggaggagggaccattagcccagagctctgagtggtttcctgttgcctgtctctaaacccctccacattcccgcggtccttcagactgcccggagagcgcgctctgcctgccgcctgcctgcctgccactgagggttcccagcaccatg

SEQ ID NO: 34 - DNA, 22 bp, Artificial sequence
  Primer
ttctgcacttaactcgtctaaa

SEQ ID NO: 35 - DNA, 22 bp, Artificial sequence
  Primer
gctgattctacgtttcaacttg

SEQ ID NO: 36 - DNA, 22 bp, Artificial sequence
  Primer
tgaggacaaggaccaggtattt

SEQ ID NO: 37 - DNA, 21 bp, Artificial sequence
  Primer
aggaaaccactcagagctctg

SEQ ID NO: 38 - DNA, 16 bp, Artificial sequence
  Primer
gtcatccctccaggcc

SEQ ID NO: 39 - DNA, 19 bp, Artificial sequence
  Primer
cggggttggtgcaactata

SEQ ID NO: 40 - DNA, 40 bp, Artificial sequence
  Primer
tgtaaaacgacggccagttgaggacaaggaccaggtattt

SEQ ID NO: 41 - DNA, 38 bp, Artificial sequence
  Primer
caggaaacagctatgacaggaaaccactcagagctctg

SEQ ID NO: 42 - DNA, 37 bp, Artificial sequence
  Primer
tgtaaaacgacggccagtggtggaggggagatagacc

SEQ ID NO: 43 - DNA, 37 bp, Artificial sequence
  Primer
caggaaacagctatgactcccaaggaactcaggacag

SEQ ID NO: 44 - DNA, 40 bp, Artificial sequence; identical to SEQ ID NO: 40
  Primer

SEQ ID NO: 45 - DNA, 38 bp, Artificial sequence; identical to SEQ ID NO: 41
  Primer

SEQ ID NO: 46 - DNA, 38 bp, Artificial sequence
  Primer
tgtaaaacgacggccagtaaatggcaccatcacaacct

SEQ ID NO: 47 - DNA, 37 bp, Artificial sequence
  Primer
caggaaacagctatgacccgaagcctgtgtcctactt

SEQ ID NO: 48 - DNA, 40 bp, Artificial sequence
  Primer
tgtaaaacgacggccagtttctgcacttaactcgtctaaa

SEQ ID NO: 49 - DNA, 39 bp, Artificial sequence
  Primer
caggaaacagctatgacgctgattctacgtttcaacttg

SEQ ID NO: 50 - DNA, 34 bp, Artificial sequence
  Primer
tgtaaaacgacggccagtgtcatccctccaggcc

SEQ ID NO: 51 - DNA, 36 bp, Artificial sequence
  Primer
caggaaacagctatgaccggggttggtgcaactata

SEQ ID NO: 52 - DNA, 23 bp, Artificial sequence
  Primer
cgaagaggaggtggtggcggaaa

SEQ ID NO: 53 - DNA, 26 bp, Artificial sequence
  Primer
ggttgttgtcctcatccctctcatac

SEQ ID NO: 54 - DNA, 24 bp, Artificial sequence
  Primer
ctctctgctcctcctgttcgacag

SEQ ID NO: 55 - DNA, 24 bp, Artificial sequence
  Primer
aggggtcttactccttggaggcca